SEQUENCE LISTING (3 sequences)

SEQ ID NO: 1 - DNA, 1134 bp, Eucalyptus Camaldulensis
  gene (1)..(1134)
atggccgtaagtttcgtaacaacatctcctgaggaagaagacaaaccgaagctaggccttggaaatattcaaactccgttaatcttcaacccttcaatgcttaaccttcaagccaatatcccaaaccaattcatctggcctgacgacgaaaaaccttccatcaacgtcctcgagcttgatgttcctctcatcgaccttcaaaaccttctctctgatccatcctccactttagatgcttcgagactgatctctgaggcctgtaagaagcacggtttcttcctcgtggtcaatcacggcatcagcgaggagcttatttcagacgctcatgaatacacgagccgcttctttgatatgcctctctccgaaaaacagagggttcttagaaaatccggtgagagtgttggctacgcaagcagtttcaccggacgcttctccaccaagcttccatggaaggagaccctttctttccggttttgcgacgacatgagccgctcaaaatccgttcaagattacttctgcgatgcgttgggacatgggtttcagccatttgggaaggtgtatcaagagtattgtgaagcaatgagttctctatcactgaagatcatggagcttctggggctaagtttaggcgtaaaacgggactactttagagagtttttcgaagaaaacgattcaataatgagactgaattactaccctccatgtataaaaccagatctcacactaggaacaggacctcattgtgatccaacatctcttaccatccttcaccaagaccatgttaatggccttcaagtctttgtggaaaatcaatggcgctccattcgtcccaaccccaaggcctttgtggtcaatatcggcgatactttcatggctctatcgaacgatagatacaagagctgcttgcaccgggcggtggtgaacagcgagagcgagaggaaatcacttgcattcttcttgtgtccgaaaaaagacagagtagtgacgccaccgagagagcttttggacagcatcacatcaagaagataccctgacttcacatggtctaagttccttgagttcactcagaaacattatagagcagacatgaacactgtccaagccttttcagattggctcaccaaacccatctaa

SEQ ID NO: 2 - DNA, 323 bp, Eucalyptus Camaldulensis
  promoter (1)..(323)
aaaggagggatccttataggggagcgggtcatttcgtgcggtaggtgagtcttggtaaacattgtctttttcccttatatatatatatatcttgagtgctagccaccttcgactcaagccgtccacaagcgtccacgagtcctgccttccctgtgcatgcccgacaagcggcccccctcctctcctcctcctcctccctcccctctccaaccaccgcccctcaaacgtcagcacctcactcctcccccacctcctgaacccctctcccccctcgccggcaatcccaccatgcccgtcgacgccctccatggttccggccaatc

SEQ ID NO: 3 - protein, 377 aa, Eucalyptus Camaldulensis
  mat_peptide (1)..(377)
MAVSFVTTSPEEEDKPKLGLGNIQTPLIFNPSMLNLQANIPNQFIWPDDEKPSINVLELDVPLIDLQNLLSDPSSTLDASRLISEACKKHGFFLVVNHGISEELISDAHEYTSRFFDMPLSEKQRVLRKSGESVGYASSFTGRFSTKLPWKETLSFRFCDDMSRSKSVQDYFCDALGHGFQPFGKVYQEYCEAMSSLSLKIMELLGLSLGVKRDYFREFFEENDSIMRLNYYPPCIKPDLTLGTGPHCDPTSLTILHQDHVNGLQVFVENQWRSIRPNPKAFVVNIGDTFMALSNDRYKSCLHRAVVNSESERKSLAFFLCPKKDRVVTPPRELLDSITSRRYPDFTWSKFLEFTQKHYRADMNTVQAFSDWLTKPI

(19) United States
(12) Patent Application Publication
(10) Pub. No.: US 2013/0117890 A1
(43) Pub. Date: May 9, 2013

(54) PROCESSES FOR ACCELERATING PLANT GROWTH AND INCREASING CELLULOSE YIELD

(71) Applicants: Sermsawat Tunlayaanukit, Bangkok (TH); Artit Vimoltust, Bangkok (TH)

(72) Inventors: Sermsawat Tunlayaanukit, Bangkok (TH); Artit Vimoltust, Bangkok (TH)

(73) Assignee: SCG PAPER PUBLIC COMPANY LIMITED, Bangkok (TH)

(21) Appl. No.: 13/511,638

(22) PCT Filed: Nov. 24, 2010
(86) PCT No.: PCT/TH10/00044

§ 371 Date: May 23, 2012

(30) Foreign Application Priority Data
Nov. 25, 2009 (TH) 0901005270

(51) Int. Cl. C12N 15/82

(52) U.S. Cl. CPC C12N 15/8246
USPC 800/290; 435/320.1; 435/419; 435/414; 435/469; 800/298; 800/317.3

(57) ABSTRACT

A polynucleotide including a gibberellin 20 oxidase gene and a promoter, more specifically a vascular specific promoter that includes one of (a) a nucleotide sequence of SEQ ID No. 2; (b) a nucleotide sequence of substantial sequence similarity to SEQ ID No. 2; (c) a nucleotide sequence that complements or is able to hybridize to (a) or (b); and (d) a nucleotide sequence which is the reverse complement of (a), (b), or (c). The gibberellin 20 oxidase gene includes one of (1) a nucleotide sequence listed in SEQ ID No. 1 as shown in FIG. 2, a fragment, domain, or feature thereof; (2) a nucleotide sequence of substantial sequence similarity to SEQ ID No. 1; (3) a nucleotide sequence that complements or is able to hybridize to (1) or (2); and (4) a nucleotide sequence which is the reverse complement of (1), (2), or (3). A vector and a transgenic plant that include the gibberellin 20 oxidase gene and the promoter are also provided by the present disclosure. Processes for generating the transgenic plant and for accelerating plant growth and/or increasing cellulose yield are also provided by various embodiments of the present disclosure.

### TECHNICAL FIELD

The present disclosure relates to the modulation of plant growth and/or quality. More particularly, the present disclosure relates to use of biotechnology and molecular biology, more specifically genetic engineering, for accelerating plant growth and increasing cellulose yield by way of modulating gibberellin 20 oxidase expression.

### BACKGROUND

Commercial forestry generally supplies the raw materials for lumber, plywood, paper, packaging, and various other wood-based materials that are essential for modern living. Global demand for wood as a raw material is increasingly at a rapid rate. However, a major disadvantage or limitation of traditional tree breeding or forestry techniques is the significantly lengthy time period that is required for trees to reach maturity. Therefore, a key focus or strategy for meeting the increasing demand for wood and other associated raw materials is to accelerate the growth rate of trees. Another useful strategy for meeting the increasing demand for wood and other associated raw materials is to enhance the quality of trees, for instance to increase the yield (e.g., cellulose yield) of trees.

Generally, plants grow via two pathways, namely (a) by apical meristems that can produce primary tissue and extend branches and roots; and (b) by a secondary growth pathway that produces new tissue from the cambium, thereby causing an extension of tree trunk circumference. The plant growth process is influenced by several factors, including environmental factors and internal signaling pathway factors such as hormones, nutrient-based signaling factors, and cell cycle or development stage dependent signaling. Typically, internal signaling pathway factors bind to receptors that are present on the cell wall(s), thereby triggering intracellular signaling.

There are many existing methods and techniques for accelerating plant growth. For example, the use of chemical fertilizers to increase or accelerate plant growth is common in agriculture. Recent developments in biotechnology and molecular biology (e.g., genetic engineering technology) have been applied to tree breeding and forestry programs for accelerating tree growth and improving wood quality and yield (Tzfira, T. et al, Trends Biotechnology, 16, page 439-446 (1998), Merkle, S. A. & Dean, J. F. D, Current Opinion on Biotechnology, 11, page 298-302 (2000), Fennin, T. M. & Gershanzon, Journal Trends Biotechnology, 20, page 291-296 (2002).

Generally, genetic engineering techniques involve identifying, extracting, and amplifying selected genes, which are associated with desired growth characteristics, for instance faster growth or phenotypic advantages. Such selected genes are used for transforming or modifying old germplasm and genome (i.e., the germplasm or genome of the organism to be transformed).

There are several patent documents disclosing the modulation (e.g., acceleration) of plant growth of multi-cellular plants using molecular biotechnology techniques. For example, international patent application WO2006/008271 teaches increasing the activity or expression of protein SnRK2, which is a kinase related to stress response, for modulating plant growth. European patent EP1580274A1 discloses increasing ribosome expression for enhancing plant growth and international patent application WO2006/018432 describes the relationship between increasing expression of protein that links with RNA and plant growth. US patent application US2008/0127365 discloses modifying an expression of nucleic acid, which is the code of type 1 homeo-domain leucine zipper polypeptide, for modulation plant growth. In addition, it has been disclosed that plant growth can be modulated by increasing the expression of LRR-receptor protein kinase (international patent application WO2006/005771A1), and increasing a cell cycle controller in A-type dependent kinase (international patent application WO2008/0134355).

The cellulose yield (i.e., ratio of cellulose to whole wood) of plants, more specifically perennial plants or trees, is an important consideration for the paper and cellulosic ethanol industries. Technologies, methods, and/or techniques for increasing the cellulose yield of trees will benefit the paper and cellulosic ethanol industries.

Cellulose is a straight chain glucan-polymer that is composed of multiple glucose molecules linked together via a β-(1,4) linkage. This straight chain glucan-polymer crystallizes into a micro fibril, which causes cellulose to be flexible and strong. FIG. 1 shows the pathway for cellulose biosynthesis. Cellulose is synthesized from glucose via carbohydrate metabolism in cellulose producing cells inside the secondary cell wall (Djerbi et al, Cellulose, 11, page 301-312, 2004). Previous attempts at increasing the quantity of cellulose include increasing the expression of CelA gene, which codes for the catalytic unit of the cellulose synthase enzyme, and increasing the expression of a homologue of the CelA gene such as RSW1.

Gibberellins (GAs) are a group of more than 100 tetracyclic diterpenes. Over the last decade, certain gibberellins have been recognized as being influential in the growth and development of plants, for example in shoot elongation, expansion and shape of leaves, and flowering and seed germination. An example illustrating the importance of gibberellins in controlling shoot elongation is the gibberellin-deficient mutants of Arabidopsis, maize, and pea (or genetically modified gibberellin-deficient Arabidopsis, maize, and pea). Gibberellin-deficient mutants of Arabidopsis, maize, and pea have reduced levels of gibberellins as compared to wild type plants, resulting in a dwarfed phenotype due to a reduction in internodal length. However, the phenotype of such gibberellin-deficient mutants (i.e., the dwarfed phenotype) can be completely restored by the application of an active gibberellin to said mutants. The function of gibberellins for accelerating plant growth and length extension of the xylem of plants has also been research. In addition, at the cellular level, gibberellins have been found to promote cell division and cell elongation.

Application of chemicals that alter gibberellin levels in plants is common in traditional agriculture and horticulture. However, in order to reduce the use of these chemicals, a biological approach such as producing a genetic modification of endogenous gibberellin biosynthesis has been suggested. Using Arabidopsis as a model organism, it has been shown that it is possible to modulate gibberellin levels in an organism (e.g., a plant) by modifying the level (or amount) of gibberellin oxidase enzyme in the organism (e.g., the plant). Increasing production of gibberellin oxidase enzymes has produced Arabidopsis plants that flower faster and have longer stems.

The modification of gibberellin biosynthesis in higher species, such as trees, would be useful in accelerating tree growth, cell compartmentalization and extension of xylem tissue, and/or increasing length and width of hardwood and softwood. Increasing the level of gibberellins in trees may also be effective in enhancing tree quality, for instance improving the cellulose yield of trees.

### SUMMARY

In accordance with a first embodiment of the present disclosure, there is disclosed a polynucleotide that includes a gibberellin 20 oxidase gene and a promoter. The promoter includes one of: (a) a nucleotide sequence of SEQ ID No. 2; (b) a nucleotide sequence of at least 70% sequence similarity to SEQ ID No. 2; (c) a nucleotide sequence that is able to hybridize to at least one of (a) and (b); and (d) a nucleotide sequence which is the reverse complement of at least one of (a), (b), and (c).

In accordance with a second embodiment of the present disclosure, there is disclosed a vector carrying a polynucleotide that includes a gibberellin 20 oxidase gene for expressing gibberellin 20 oxidase polypeptide and a promoter for controlling the expression of the gibberellin 20 oxidase gene. The promoter includes one of: (a) a nucleotide sequence, of SEQ ID No. 2; (b) a nucleotide sequence of at least 70% sequence similarity to SEQ ID No. 2; (c) a nucleotide sequence that is able to hybridize to at least one of (a) and (b); and (d) a nucleotide sequence which is the reverse complement of at least one of (a), (b), and (c).

In accordance with a third embodiment of the present disclosure, there is disclosed a transgenic plant cell that includes a polynucleotide that includes a gibberellin oxidase gene for expressing a gibberellin 20 oxidase polypeptide and a promoter for controlling expression of the gibberellin oxidase gene. The promoter includes one of: (a) a nucleotide sequence of SEQ ID No. 2; (b) a nucleotide sequence of at least 70% sequence similarity to SEQ ID No. 2; (c) a nucleotide sequence that is able to hybridize to at least one of (a) and (b); and (d) a nucleotide sequence which is the reverse complement of at least one of (a), (b), and (c).

In accordance with a fourth embodiment of the present disclosure, there is disclosed a process for generating a transgenic plant cell via a gene transformation technique using an agro-bacteria to transfer a polynucleotide into a plant cell. The polynucleotide that is transferred or introduced into the plant cell to thereby produce the transgenic plant cell includes a gibberellin 20 oxidase gene and a promoter. The promoter includes a nucleotide sequence of at least 70% sequence similarity to SEQ ID No. 2.

In accordance with a fifth embodiment of the present disclosure, there is disclosed a process for modulating plant growth. The process includes transfecting a plant with a polynucleotide to produce a transgenic plant. The transgenic plant includes a gibberellin 20 oxidase gene and a promoter. The promoter includes a nucleotide sequence of at least 70% sequence similarity to SEQ ID No. 2. The process further includes expressing the gibberellin 20 oxidase gene in the plant to produce gibberellin 20 oxidase polypeptide.

In accordance with a sixth embodiment of the present disclosure, there is disclosed a transgenic perennial plant that includes a polynucleotide that includes a gibberellin oxidase gene for expressing a gibberellin 20 oxidase polypeptide and a promoter for controlling expression of the gibberellin oxidase gene. The promoter includes one of: (a) a nucleotide sequence of SEQ ID No. 2; (b) a nucleotide sequence of at least 70% sequence similarity to SEQ ID No. 2; (c) a nucleotide sequence that is able to hybridize to at least one of (a) and (b); and (d) a nucleotide sequence which is the reverse complement of at least one of (a), (b), and (c).

Various embodiments of the present disclosure disclose, are associated with, or relate to increase in expression of gibberellin 20 oxidase. In certain embodiments, the increase in gibberellin 20 oxidase production in transgenic perennial plants, for example eucalypt plants (e.g., Eucalyptus plant) and Tobacco plants, can facilitate or effectuate an increased cellulose production and yield in said transgenic perennial plants. An increased cellulose production in transgenic perennial plants can facilitate or effectuate an increased paper yield or production associated with said transgenic perennial plants.

### BRIEF DESCRIPTION OF THE FIGURES

The present disclosure is described with reference to the figures, in which:

FIG. 1 shows a pathway for cellulose biosynthesis;

FIG. 2 shows SEQ ID NO. 1, which is a polynucleotide sequence encoding for gibberellin 20 oxidase;

FIG. 3 shows SEQ ID NO. 2, which is a polynucleotide sequence of a promoter from the species Eucalyptus Camaldulensis;

FIG. 4 shows SEQ ID NO. 3, which represents a polypeptide sequence of a gibberellin 20 oxidase polypeptide;

FIG. 5 shows a vector map for expressing a gibberellin 20 oxidase gene according to an embodiment of the present disclosure;

FIG. 6 is a graph showing increased growth with a transgenic plant as compared to a wild type plant, the transgenic plant including a gibberellin 20 oxidase gene and a promoter according to an embodiment of the present disclosure;

FIG. 7 is a picture showing increased growth of transgenic plants, which include a gibberellin 20 oxidase gene and a promoter of particular embodiments of the present disclosure, as compared to wild type plants;

FIG. 8 is a graph showing increased cellulose production in a transgenic plant as compared to a wild type plant, the transgenic plant including a gibberellin 20 oxidase gene and a promoter according to an embodiment of the present disclosure; and

FIG. 9 is a photo of a gel electrophoresis showing the existence of a gibberellin 20 oxidase gene and a promoter as provided by various embodiments of the present disclosure in transfected plant tissue.

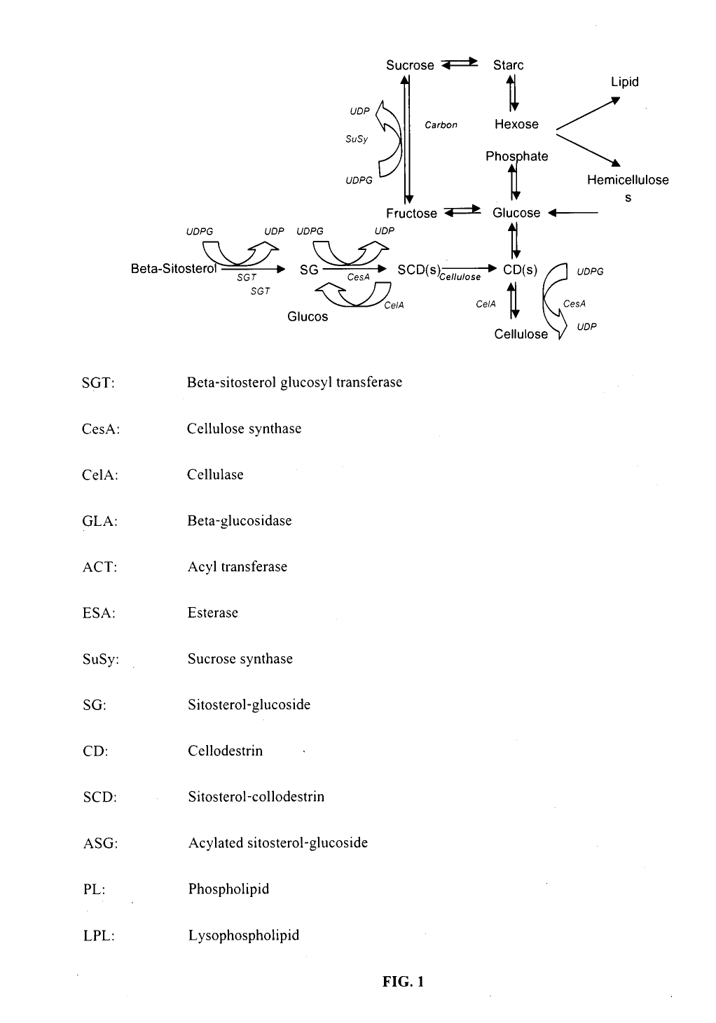

### DETAILED DESCRIPTION

The present disclosure discloses that gibberellin 20 oxidase is involved in modulation of plant growth, more specifically acceleration of plant growth and/or increase cellulose production. The present disclosure also discloses that increased production of gibberellin 20 oxidase can facilitate or effectuate an increased and/or accelerated plant growth. The present disclosure also provides a promoter, more specifically a vascular specific promoter, that is able to modulate gibberellin 20 oxidase production in plant vascular tissue. More specifically, the vascular specific promoter can facilitate or effectuate an increased gibberellin 20 oxidase production specifically in plant vascular tissue, thereby enabling at least one of accelerated plant growth and enhanced cellulose production (or yield).

Aspects of the present disclosure relate to isolated nucleic acids, or polynucleotides, that include a coding sequence for gibberellin 20 oxidase (also referred to as gibberellin 20 oxidase polypeptide, enzyme, or protein). The coding sequence for gibberellin 20 oxidase can be referred to as a gibberellin 20 oxidase gene.

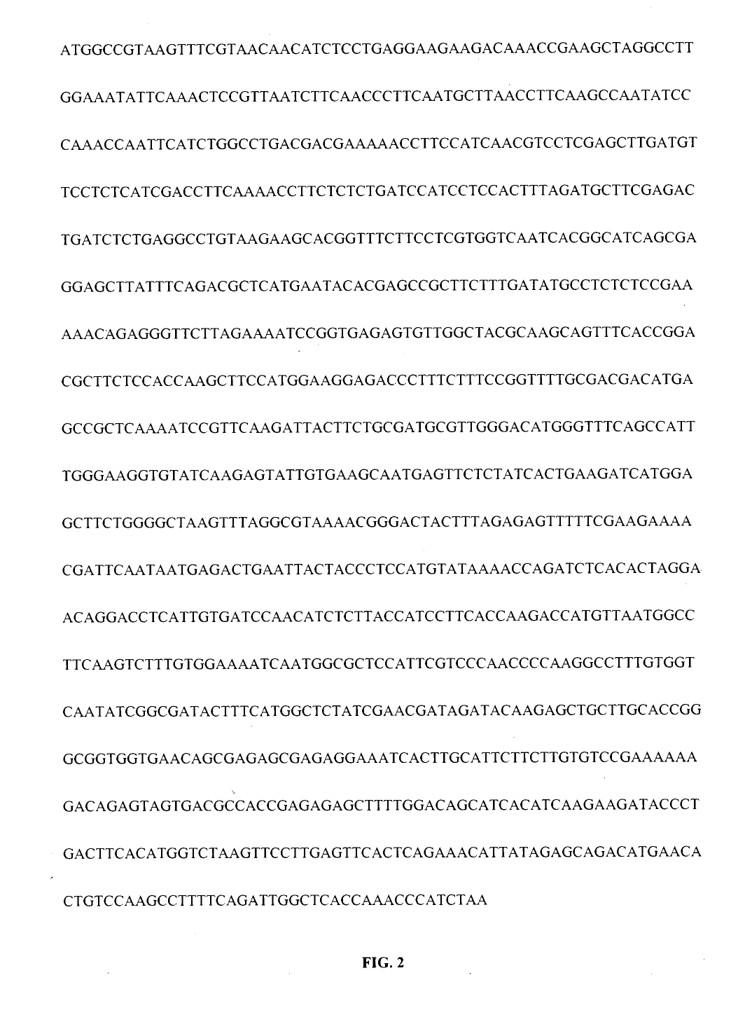

Many embodiments of the present disclosure relate to a gibberellin 20 oxidase gene that includes at least one of (1) a nucleotide sequence listed in SEQ ID No. 1 as shown in FIG. 2, a fragment, domain, or feature thereof; (2) a nucleotide sequence of substantial sequence similarity to SEQ ID No. 1; (3) a nucleotide sequence that complements or is able to hybridize to (1) or (2); and (4) a nucleotide sequence which is the reverse complement of (1), (2), or (3).

Aspects of the present disclosure also relate to nucleic acids, or polynucleotides, that include a promoter or promoter region. The promoter can modulate the expression of the gibberellin 20 oxidase gene, for instance increase the expression of the gibberellin 20 oxidase gene. In many embodiments, the promoter is a vascular specific promoter. The vascular specific promoter can specifically facilitate, direct, or effectuate an increased and/or accelerated expression of the gibberellin 20 oxidase gene, and consequent production of gibberellin 20 oxidase, in plant vascular tissue (e.g., xylem tissue). The increased production of gibberellin 20 oxidase in plant vascular tissue (e.g., xylem tissue) can facilitate or effectuate an increased in length of plant xylem tissue and enhanced cellulose production (or yield).

In many embodiments, the promoter is obtained from a perennial plant. In several embodiments, the promoter is obtained or extracted from a Eucalypt, for example a Eucalyptus plant or tree. More specifically, in various embodiments, the promoter is obtained or extracted from the species Eucalyptus Camaldulensis.

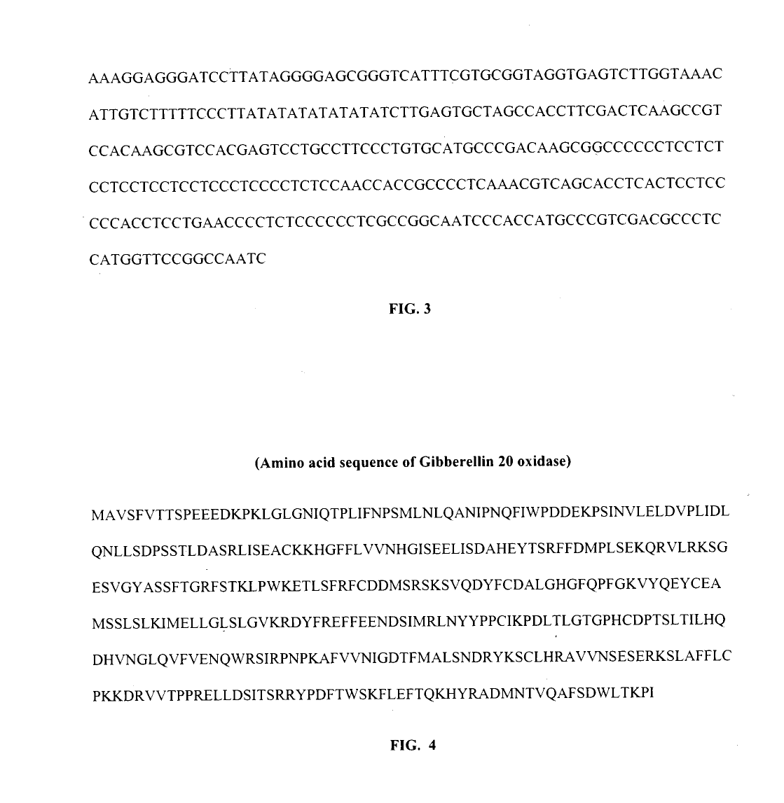

The promoter includes one of: (a) a nucleotide sequence of SEQ ID No. 2 as shown in FIG. 3; (b) a nucleotide sequence of substantial sequence similarity to SEQ ID No. 2; (c) a nucleotide sequence that complements or is able to hybridize to (a) or (b); and (d) a nucleotide sequence which is the reverse complement of (a), (b), or (c).

Many embodiments of the present disclosure provide nucleic acids, or polynucleotides, that include both the gibberellin 20 oxidase gene as well as the promoter. Expression of the gibberellin 20 oxidase gene produces gibberellin 20 oxidase and the promoter modulates the expression of the gibberellin 20 oxidase gene. The promoter includes one of: (1) a nucleotide sequence listed in SEQ ID No. 1 as shown in FIG. 2; (2) a nucleotide sequence of substantial sequence similarity to SEQ ID No. 1; (3) a nucleotide sequence that complements or is able to hybridize to (1) or (2); and (4) a nucleotide sequence which is the reverse complement of (1), (2), or (3). The gibberellin 20 oxidase gene includes one of: (a) a nucleotide sequence of SEQ ID No. 2 as shown in FIG. 3; (b) a nucleotide sequence of substantial sequence similarity to SEQ ID No. 2; (c) a nucleotide sequence that complements or is able to hybridize to (a) or (b); or (d) a nucleotide sequence which is the reverse complement of (a), (b), or (c).

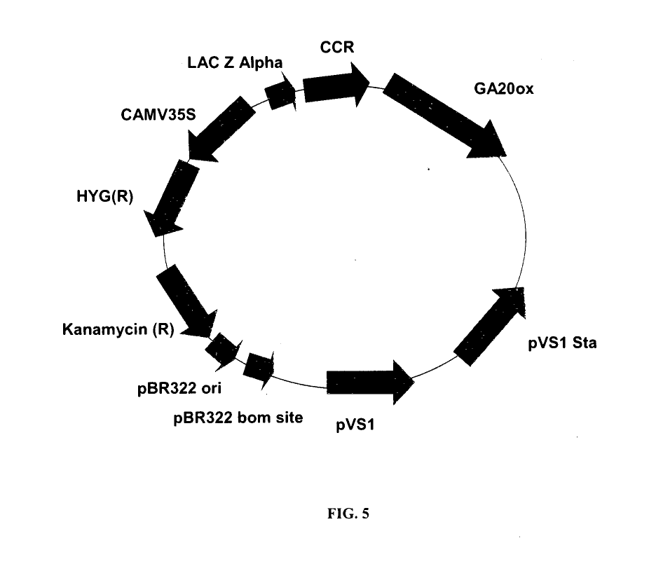

Expression of the gibberellin 20 oxidase gene produces gibberellin 20 oxidase polypeptide. In various embodiments, the gibberellin 20 oxidase polypeptide includes (i) an amino acid sequence of SEQ ID No. 3 as shown in FIG. 4; (ii) an amino acid sequence of substantial similarity of SEQ ID No. 3; or (iii) an amino acid sequence that complements or is able to hybridize to (i) or (ii).

The term “substantial similarity” for purposes of the present disclosure shall be understood to be at least approximately 70%, preferably at least approximately 75% similar, more preferably at least approximately 80% similar, and even more preferably at least approximately 90% similar to a particular nucleotide sequence or amino acid sequence.

### Polynucleotides

Polynucleotides can be generally defined as a polymeric compound, usually DNA (deoxyribonucleic acid) or RNA (ribonucleic acid) that includes multiple nucleotides that are covalently bonded in a chain. The term “nucleic acids” or “polynucleotides” as referred to in this disclosure may refer to nucleic acids or polynucleotides that are present in nature (e.g., naturally present in living organism) that can be, or are, isolated from the living organism by using a process disclosed in this disclosure or by any other processes (e.g., processes for extracting or obtaining polynucleotides from organisms) that are known in the art. Alternatively, the term “nucleic acids” or “polynucleotides” as referred to in this disclosure may be wholly or partially synthetic. For example, the polynucleotides can be synthesized artificially or directly, e.g., by using an automated synthesizer. Alternatively, the polynucleotides can be recombinant polynucleotides, which means that particular sections of nucleotides (or nucleotide sequences) do not run contiguously in nature. Such sections of nucleotides have been ligated separately and subsequently combined artificially.

A variant polynucleotide sequence may be a mutant, a homologue, or an allele of a particular polynucleotide sequence, for instance polynucleotide sequences provided by the present disclosure such as the polynucleotide sequence of SEQ ID No. 1 or the polynucleotide sequence of SEQ ID No. 2. Changes to a particular polynucleotide sequence can occur by one or more of addition, insertion, deletion, and substitution of one or more nucleotides of said polynucleotide sequence, thereby leading to an addition, insertion, deletion, or substitution of one or more amino acids in the encoded or expressed polypeptide from said polynucleotide sequence.

The present disclosure further discloses or teaches that the expression of the gibberellin 20 oxidase gene, and consequent production of gibberellin 20 oxidase, can be controlled, modulated, and/or changed (e.g., increased) by use of promoters (or promoter sequences), more specifically the promoter as provided by various embodiments of the present disclosure.

In many embodiments, the promoter facilitates or effectuates an increased expression of the gibberellin 20 oxidase gene, and consequent production of gibberellin 20 oxidase. In several embodiments, the promoter increases the expression of gibberellin 20 oxidase gene, and consequent production of gibberellin 20 oxidase, specifically in plant vascular tissue. The promoter can facilitate or effectuate a specific, exclusive, or substantially exclusive expression of the gibberellin 20 oxidase gene, and consequent production of gibberellin 20 oxidase, in plant vascular tissue, for example xylem tissue.

### Vascular Tissue

As mentioned above, the vascular specific promoter provided by various embodiments of the present disclosure can direct expression or production of gibberellin 20 oxidase to vascular tissue, such as the xylem, phloem, or cambium.

Vascular tissue is a complex conducting tissue and is generally created in two life cycles of a plant, more specifically during embryogenesis and post-embryogenesis. The primary components of vascular tissue are the xylem and the phloem. The vascular tissue also includes two meristems, namely the vascular cambium and the cork cambium. The vascular tissue in plants is arranged in discrete strands called vascular bundles. The xylem is typically disposed closer the core or interior of the stem (or tree trunk) with the phloem disposed towards the exterior or circumference of the stern (or tree trunk). The vascular cambium is located between the xylem and phloem. In trees (or plants that develop wood), the vascular cambium divides off cells that will add to the xylem and phloem tissue thereby increasing the girth of the plant and leading to increased production of wood. The cork cambium will give rise to thickened cork cells for protecting the surface of the plant. The production of wood and the production of cork represent secondary growth of the plant.

The xylem transports water and minerals from roots to stems and leaves. Both primary and secondary xylem contain trachea, fiber, and parenchyma. Trachea is composed of trachids and perforated tubes that are responsible for water delivery. The xylem includes fibers that strengthen and provide the durability to the vascular tissue. The parenchyma cells of the xylem include radial cells and longitudinal cells that are responsible for storing starch, oil, and other energy-providing molecules.

The phloem transports products synthesized by photosynthesis, which generally occurs in the leaves of the plants, to other parts of the plant. The phloem includes sieve elements, sieve tubes, parenchyma cells, and sieve element fibers. Sieve tubes include multiple tubes that combine longitudinally and connect to each other through the cell wall. The phloem also includes assembly cells and albuminous cells that are responsible for storing proteins and substances that are important in the function of the phloem. The parenchyma cells of the phloem control the storage of starch, fats, and other chemicals or components. Phloem fibers are important for strengthening the phloem and for accumulating or storing starch in the plant.

### Vectors

Various embodiments of the present disclosure also relate to a vector as shown in FIG. 5. The vector includes a polynucleotide as provided by various embodiments of the present disclosure.

In many embodiments, the vector includes a polynucleotide that includes the gibberellin 20 oxidase gene and the promoter. This means that vector includes both the gibberellin 20 oxidase gene, which includes one of (1) the nucleotide sequence of SEQ ID No. 1; (2) a nucleotide sequence of substantial sequence similarity to SEQ ID No. 1; (3) a nucleotide sequence that complements or is able to hybridize to (1) or (2); and (4) a nucleotide sequence which is the reverse complement of (1), (2), or (3); and the promoter, which includes one of (a) a nucleotide sequence of SEQ ID No. 2; (b) a nucleotide sequence of substantial sequence similarity to SEQ ID No. 2; (c) a nucleotide sequence that complements or is able to hybridize to (a) or (b); and (d) a nucleotide sequence which is the reverse complement of (a), (b), or (c).

Vectors generally serve as carriers for transfer or insertion of gene(s) (i.e., polynucleotides). Vectors that carry or include a specific, more particularly a desired, gene or polynucleotide are typically inserted or introduced into a host organism. The gene or polynucleotide that is introduced into the host organism by the vector can be expressed within the host organism to produce polypeptides or proteins (e.g., enzymes).

The choice of vector for carrying the polynucleotide that includes both the first coding sequence and the second coding sequence as described above can be dependent upon a number of factors. For example, the vector selected should be suitable or compatible with the host cell that the vector will be introduced intro. In various embodiments of the present disclosure, the pCAMBIA plasmid is used as the vector, i.e., for carrying the polynucleotide of the present disclosure. The assembly of the polynucleotide within the genome of the vector can occur by connecting cohesive sections of polynucleotides or DNA. The assembly of polynucleotides into existing genomes can occur by a technique or method as disclosed in the present disclosure (e.g., in the examples below) or by another technique or method that is known in the art. As an example, restriction enzymes can be used for creating complementary connection points. Different DNA or polynucleotide fragments with such complementary connections points (or ends) can interconnect together to thereby form a continuous polynucleotide sequence.

### Transgenic Plants/Cells/Tissue

Various embodiments of the present disclosure relate to plants, cells, or tissues, more specifically transgenic plants, cells, or tissues, that are genetically modified using the vector as provided by various embodiments of the present disclosure.

The transgenic plants, cells, and tissues include the polynucleotide of particular embodiments of the present disclosure, wherein the polynucleotide is introduced into said transgenic plants, cell, and tissues with the use of said vectors. According, the transgenic plants, cells, and tissues include the gibberellin 20 oxidase gene and the promoter.

The transgenic plants, cells, and tissues include both the gibberellin 20 oxidase gene, which includes one of (1) the nucleotide sequence of SEQ ID No. 1; (2) a nucleotide sequence of substantial sequence similarity to SEQ ID No. 1; (3) a nucleotide sequence that complements or is able to hybridize to (1) or (2); and (4) a nucleotide sequence which is the reverse complement of (1), (2), or (3); and the promoter, which includes one of (a) a nucleotide sequence of SEQ ID No. 2; (b) a nucleotide sequence of substantial sequence similarity to SEQ ID No. 2; (c) a nucleotide sequence that complements or is able to hybridize to (a) or (b); and (d) a nucleotide sequence which is the reverse complement of (a), (b), or (c).

The transgenic plant can be a perennial plant. For instance the transgenic plant can be a Eucalyptus plant (e.g., a plant of the species Eucalyptus Camaldulensis). Alternatively, the transgenic plant can be a Tobacco plant (e.g., a plant of the species Nicotiana tabacum). It will be understood that the polypeptide of various embodiments of the present disclosure can be introduced into other perennial plants, for instance wood-producing plants, for producing corresponding transgenic plants within the scope of the present disclosure.

The transgenic plant, which includes both the gibberellin 20 oxidase gene and the promoter, can have an increased and/or accelerated growth as compared to wild-type plants (i.e., plants that do not include the gibberellin 20 gene or the promoter). The increase and/or acceleration in growth of the transgenic plant can be due to an increase in gibberellin 20 oxidase production in said transgenic plant due to an increased expression of gibberellin 20 oxidase, which can be facilitated or effectuated by the promoter.

In addition, the transgenic plant can have, or demonstrate, an increased cellulose production, and hence cellulose yield. The promoter can facilitate or effectuate increased expression of the gibberellin 20 oxidase gene, and hence production of gibberellin 20 oxidase, specifically in the vascular tissue of the plant, thereby enabling the increased cellulose yield.

### Aspects of a Process for Generating a Transgenic Plant

The present disclosure also relates to processes for generating the transgenic plant. The process includes transforming plants with the use of vectors, more specifically vectors provided by various embodiments of the present disclosure. Transformation of plants involves introduction of new (i.e., not originally present) polynucleotides or genes into said plants. Various transformation techniques or processes are known in the art, and can be applied or used with the processes of the present disclosure.

The generated or produced transgenic plants, cells, and tissues include both the gibberellin 20 oxidase gene, which includes one of (1) the nucleotide sequence of SEQ ID No. 1; (2) a nucleotide sequence of substantial sequence similarity to SEQ ID No. 1; (3) a nucleotide sequence that complements or is able to hybridize to (1) or (2); and (4) a nucleotide sequence which is the reverse complement of (1), (2), or (3); and the promoter, which includes one of (a) a nucleotide sequence of SEQ ID No. 2; (b) a nucleotide sequence of substantial sequence similarity to SEQ ID No. 2; (c) a nucleotide sequence that complements or is able to hybridize to (a) or (b); and (d) a nucleotide sequence which is the reverse complement of (a), (b), or (c).

The transgenic plant can be a perennial plant, for instance a Eucalyptus plant, a Tobacco plant, or other perennial plants as indicated above.

Aspects of a Process for Accelerating Plant Growth and/or Increasing Cellulose Yield of Transgenic Plants

A process for accelerating plant growth and/or increasing cellulose yield of transgenic plants is also provided according to various embodiments of the present disclosure.

The process includes generating a transgenic plant by using processes for generating transgenic plants provided by various embodiments of the present disclosure. A plant, for example a perennial plant such as a Eucalyptus plant, can be transformed (or genetically modified) by using vectors. In the present disclosure, the transformation of plants involves the introduction of the gibberellin 20 oxidase gene and the promoter into said plant's genome. Transformation of plants can occur via various techniques or processes that are known in the art.

The process for accelerating plant growth and/or increasing cellulose yield of transgenic plants further includes expressing the gibberellin 20 oxidase gene in the transgenic plant.

As above described, the gibberellin 20 oxidase gene includes one of: (1) the nucleotide sequence of SEQ ID NO. 1; (2) the nucleotide sequence of substantial sequence similarity to SEQ ID No. 1; (3) the nucleotide sequence that complements or is able to hybridize to (1) or (2); and (4) a nucleotide sequence which is the reverse complement of (1), (2), or (3).

The gibberellin 20 oxidase produced or expressed includes one of: (i) an amino acid sequence of SEQ ID No. 3; (ii) an amino acid sequence of substantial similarity of SEQ ID No. 3; or (iii) an amino acid sequence that complements or is able to hybridize to (i) or (ii).

The promoter facilitates or effectuates an increased expression of gibberellin oxidase gene, and consequent production of gibberellin 20 oxidase, in the transgenic plant as compared to wild-type plants (i.e., plants which do not include either the gibberellin 20 oxidase gene or the promoter). As described above, the promoter includes one of: (a) the nucleotide sequence of SEQ ID No. 2; (b) a nucleotide sequence of substantial sequence similarity to SEQ ID No. 2; (c) a nucleotide sequence that complements or is able to hybridize to (a) or (b); and (d) a nucleotide sequence which is the reverse complement of (a), (b), or (c).

In some embodiments, the promoter facilitates or effectuates an at least 10% increased production of gibberellin 20 oxidase in the transgenic plant as compared to non-transgenic or wild-type plants. In various embodiments, the promoter facilitates or effectuates an at least 25% increased production of gibberellin 20 oxidase in the transgenic plant as compared to wild-type plants. In specific embodiments, the promoter facilitates or effectuates an at least 40% increased production of gibberellin 20 oxidase in the transgenic plant as compared to wild-type plants.

The increase in gibberellin 20 oxidase production results in an increased or accelerated plant growth and/or an increased cellulose production in the transgenic plant as compared to wild-type plants.

In some embodiments, the promoter facilitates or effectuates specific expression of the gibberellin 20 oxidase gene, and production of gibberellin 20 oxidase, in plant vascular tissue (e.g., xylem tissue). The increased production of gibberellin 20 oxidase specifically in plant vascular tissue (e.g., xylem tissue) can effectuate an accelerated growth of plant vascular tissue (e.g., xylem tissue). For example, the increased production of gibberellin 20 oxidase specifically in xylem tissue can result in an accelerated extension in length of the xylem and/or increased cellulose production associated with the transgenic plant.

The increase in the expression of the gibberellin 20 oxidase gene, and subsequent production of gibberellin 20 oxidase, observed in association with various embodiments of the present disclosure is significant and unexpected. More specifically, the increase in the expression of gibberellin 20 oxidase gene, and subsequent production of gibberellin 20 oxidase, achieved with the use of the promoter is significant and unexpected. In addition, the ability of the promoter to specifically direct expression of the gibberellin 20 oxidase gene, and consequent production of gibberellin 20 oxidase, to plant vascular tissue is unexpected and surprising.

In addition, the ability of the promoter to specifically direct expression of the gibberellin 20 oxidase gene, and consequent production of gibberellin 20 oxidase, to plant vascular tissue represents a significant advantage as it facilitates or enables specific acceleration in vascular tissue growth.

### Gibberellin 20 Oxidase Polypeptides

Various aspects of the present disclosure relate to proteins or polypeptides with the ability to perform tasks similar, or substantially similar, to the gibberellin 20 oxidase (or gibberellin 20 oxidase 20 polypeptide) provided by various embodiments of the present disclosure. Such proteins or polypeptides, or at least a part thereof, have at least 70% similar amino acid sequence, preferably at least 80% similar amino acid sequence, and more preferably at least 95% similar amino acid sequence, to SEQ ID No.3.

For purposes of the present disclosure, the sequence identity, and hence similarity of polypeptide sequences, can be defined with reference to the algorithm GAP (Wisconsin Package, Accelerys, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4.

Other algorithms besides GAP can also be used, for example BLAST (which uses the method of Altschul et al, Journal of Molecular Biology, 215, page 405-410 (1990)), FASTA (which uses the method of Pearson and Lipman, PNAS USA, 85, page 2444-2448 (1988)), or the Smith-Walternan algorithm described in Smith and Waterman, Journal of Molecular Biology, 147, pages 195-197 (1981).

A variant polypeptide of the present disclosure can differ from the sequence of SEQ ID No. 3 by one or more of addition, insertion, addition, or substitution, of one or more amino acids. For example, a protein or polypeptide including a replacement, addition, or deletion of any amino acid in SEQ ID No.3 while still maintaining the same protein function is included in the present disclosure. Examples of substitution include from a non-polar amino acid (e.g., alanine, glycine, leucine, isoleucine, valine, proline, phenylalanine, and methionine) to an amino acid with an aromatic ring (e.g., phenylalanine, hisidine, tryptophan and tyrosine), from a neutral amino acid (e.g., alanine, asparagine, proline, serine, isoleucine, luecine, cysteine, tyrosine, and glutamine) to an acidic amino acid with a positive charge (e.g., arginine, lysine, histidine). Other substitutions that are known in the art are also included in the present disclosure, for example a change from lysine to arginine to maintain a positive charge, a change of glutamic acid to aspartic acid to maintain the negative charge, or a change from serine to threonine to maintain the hydroxyl (OH) group. In addition, amino acid substitution can also occur to introduce amino acids that have specific characteristics, for instance the introduction of cysteine can create disulphide access points.

Many embodiments of the present disclosure relate to polynucleotides that include the first coding sequence that include the first coding sequence encoding for gibberellin 20 oxidase and the second coding sequence coding for the vascular specific promoter, wherein the first coding sequence includes at least one of (1) a nucleotide sequence listed in SEQ ID No. 1 as shown in FIG. 2, a fragment, domain, or feature thereof; (2) a nucleotide sequence of substantial sequence similarity to SEQ ID No. 1; (3) a nucleotide sequence that complements or is able to hybridize to (1) or (2); and (4) a nucleotide sequence which is the reverse complement of (1), (2), or (3); and the second coding sequence includes a nucleic acid or polynucleotide of a nucleotide sequence including: (a) a nucleotide sequence of SEQ ID No. 2 as shown in FIG. 3; (b) a nucleotide sequence of substantial sequence similarity to SEQ ID No. 2; (c) a nucleotide sequence that complements or is able to hybridize to (a) or (b); or (d) a nucleotide sequence which is the reverse complement of (a), (b), or (c).

In many embodiments, the expression of said polynucleotide provided by various embodiments in a particular plant (e.g., a tree or other wood-producing plant) results in production of increased or higher levels of gibberellin 20 oxidase in said plant (e.g., tree or other wood-producing plant). This increase in the level or amount of gibberellin 20 oxidase is at significant, surprising, and/or unexpected. In several embodiments, the level of gibberellin 20 oxidase is at least approximately 10%, more preferably at least approximately 25%, and even more preferably at least approximately 40%, more than the level of gibberellin 20 oxidase in existing plants (e.g., trees or other wood-producing plants).

In addition, the expression of said polynucleotide provided by various embodiments of the present disclosure can further result in increased levels or amount of gibberellin 20 oxidase specifically in the vascular tissue of plants, for example xylem. The increased level of gibberellin 20 oxidase in specific or targeted plant tissue, here the vascular tissue (e.g., xylem), is a novel, significant, and unexpected disclosure made by the present disclosure. In addition, the increased level of gibberellin 20 oxidase in the vascular tissue according to various embodiments is at least approximately 10%, more preferably at least approximately 25%, and even more preferably at least 40%, more than the level of gibberellin 20 oxidase present in the vascular tissue of existing plants.

### EXAMPLES

### Experimental Protocol

An example of an experimental protocol associated with the present disclosure is provided below. The disclosed experimental protocol is for the purposes of illustration and does not limit the scope of the present disclosure. A person of ordinary skill in the art will understand that modified, revised, or improved techniques, methods, or process that are capable of achieving a similar, or substantially similar, result or effect are also included within the scope of the present disclosure.

### DNA Extraction

DNA is extracted from a plant, for instance from a leaf of the plant, using Cetyltrimethylammonium bromide (CTAB) plant DNA extraction. An example of the protocol for CTAB plant DNA extraction can be found at http://www.protocol-online.org/

For example, a leaf weighing approximately one gram is crushed in liquid nitrogen with a small amount of polyvinyl pyrrolidone (PVP). The mixture is then incubated in a solution including approximately 2% CTAB, NaCl of a concentration of approximately 1.4 M, 20 mM of EDTA at pH 8.0, 100 mM of Tris-HCl at pH 8.0, and 2-mercaptone ethanol with a concentration of approximately 20.6%.

The mixture is then incubated at a temperature of approximately 65° C. for 15 minutes. Subsequently, approximately 300 ml of potassium acetate at a concentration of approximately 5.0 M is added and the resultant mixture is incubated on ice for approximately 30 to 60 minutes. The mixture is then centrifuged at 13,000 rpm at 4° C. for approximately 30 minutes. The clear solution floating on the top is separated and approximately 0.7 ml of an alcoholic mixture of chloroform: isoamyl at 24:1 by volume is added to the separated top clear solution. The resultant mix is then shaken gently by using a mechanical shaker for about 20 minutes and then centrifuge at 13,000 rpm at 4° C. for approximately 30 minutes.

Again, the solution floating on the top is separated and approximately 600 ml-0.750 ml of ethanol is added to the separated top solution before incubating the resultant mixture at −20° C. for approximately 30 minutes. Subsequently, the mixture is centrifuge at approximately 13,000 rpm at 4° C. for 10 minutes.

The pellet formed is rinsed with approximately 500 ml of ethanol (70%). Then, the mixture of pellet and ethanol is centrifuged at approximately 13,000 rpm at 4° C. for 5 minutes. The resultant pellet is dried at 37° C. until the pellet is completely dry. The dry pellet is dissolved in TE solution (Tris-HCl of pH 8.0 and a concentration of approximately 10 M and EDTA of pH 8.0 and a concentration of approximately 1.0 M) and stored at −20° C. for future use.

Typically, when a particular gene needs to be isolated from genomic DNA, the process starts with cutting the genomic DNA into pieces by using enzymes, for example DNAase, with manganese or by processing by sonication. The DNA pieces are then separated according to size using agarose gel electrophoresis. The specific DNA pieces or fragment that include desired gene (e.g., the gibberellin 20 oxidase gene) can be identified using nucleic acid hybridization. A polynucleotide with a complementary sequence to that of gibberellin 20 oxidase (e.g., with a sequence similarity of at least 70% to SEQ ID. No. 1) is used as a probe.

When a DNA piece or fragment including the gibberellin 20 oxidase gene is found, the inventor amplifies identified DNA piece or fragment (or polynucleotide) using polymerase chain reaction (PCR) process before cloning the DNA fragment (or polynucleotide) into a vector.

A method for extracting or obtaining a desired polynucleotide (e.g., gibberellin 20 oxidase gene) according to various embodiments of the present disclosure involves separation of mRNA from tissues of Arabidopsis and a process of reverse PCR for creating cDNA of the gibberellin 20 oxidase before cloning cDNA into pCAMBIA plasmid (or vector). The pCAMBIA plasmid (or vector) carrying the polynucleotide of particular embodiments are used to transform bacteria cells (e.g., ago-bacterial cells) using techniques or methods known in the art.

### Amplification of the Promoter of Gene Chinnamyol CoA Reductase (CCR) Using Polymerase Chain Reaction

The promoter of a Chinnamyol CoA Reductase (CCR) gene, which is a gene from the Eucalyptus tree with 323 base pairs, is cloned using the polymerase chain reaction technique. The experimental protocol for PCR is known in the art. For example, the components for PCR can include 40 mg of DNA from the Eucalyptus tree, a primer with the concentration of 10 picomole per microliter and a volume of 3.0 microliter, 2.0 microliter of PCR solution, and 5.0 microliters of dNTPs at a concentration of 10 times dNTPs, 1.0 microliter of DNA polymerase of 5 units per microliter, and water, made to a total volume of 50 micro liters.

The polymerase chain reaction is initiated by a standard program at 94° C. for 4 minutes for 1 cycle, followed by 30 cycles of 94° C. for 1 minute, 60° C. for 1 minute, and 72° C. for 30 seconds, followed by 72° C. for 10 minutes for 1 cycle. The results of the polymerase chain reaction are checked by the electrophoresis technique using agarose Gel 0.8%, followed by a dye using ethidium bromide and checking the UV absorption. Ethidium bromide binds to DNA molecules, thereby allowing detection of the presence of specific DNA molecules (e.g., DNA molecules of specific molecular weights).

Amplification of DNA or Polynucleotides Coding for Gibberellin 20 Oxidase (i.e., the Gibberellin 20 Oxidase Gene) from the RNA of Arabidopsis

RNA is extracted from Arabidopsis using RNA extraction techniques known in the art. In an example, Arabidopsis grown in tissue culture is crushed. The crushing of Arabidopsis occurs in liquid nitrogen. RNA is extracted from the crushed sample by using RNeasy Mini Kit (QIAGEN) following the instructions provided by the product manufacturer.

cDNA is synthesized from RNA of Arabidopsis by using approximately 2.0 micro liters of RNA PrimerOligoDT at a concentration of approximately 10 picomole per microliter at a volume of 4.0 microliter 10× solution 4.0 microliter 5 mM dNTPs 4.0 microliter the extract agent of RNase 40 unit per micro liter reverse, transcript test (4 unit per micro liter) 2.0 micro liter and water 23.5 micro liter all together for the total volume of 40 micro liter. The synthesis of cDNA from RNA of Arabidopsis is performed over 1 hour at 37° C.

Gibberellin 20 Oxidase is synthesized from RNA of Arabidopsis (which has 1,134 base pairs). Polymerase chain reaction (PCR) is performed to amplify the cDNA of Arabidopsis using 4.0 micro liter (40 nano grams) of primer at a concentration of approximately 10 pM per micro liter with the volume of 1.0 micro liter 10×PCR solution with MgCl₂ 15 mM. 5.0 micro liter 1 mM. DNTPS 5.0 micro liter enzyme 5 unit per micro liter 1.0 micro liter and water 33.0 micro liter with the total volume of 50.0 micro liter. The PCR can be performed using known conditions in the art, for example a set or standard PCR program that includes 1 cycle of 94° C. for 4 minutes, 30 cycles of at 94° C. for 1 minute, and 55° C. for 1 minute, and 72° for 1 minute 20 seconds, and 1 cycle of 72° C. for 10 minutes.

The results of the polymerase chain reaction can be checked using the electrophoresis technique (e.g., gel electrophoresis) at 0.8% agarose gel, followed by application of a dye of ethidium bromide. Ethidium bromide binds to DNA molecules, thereby enabling detection of DNA molecules via UV absorption.

### Plant Vector Construction

The polynucleotide sequences, which are produced through the PCR process, are ligated or introduced into plasmids (or vectors) using techniques known in the art. A number of references describe techniques and methods for constructing plant vectors, namely Walden, R., Koncz, C. & Schell, J., Methods Molecular Cell Biology 1, 175-194 (1990) and Coles J. P. et al., Modification of gibberellin production and plant development in Arabidopsis by sense and antisense expression of gibberellin 20-oxidase genes, Plant J., 17, page 547-556 (1990).

Transformation of E. coli Bacteria

Plasmids carrying the polynucleotide sequences are transferred into E. coli bacteria by using a transformation technique or method that is commonly known in the art. Competent E. coli cells can be prepared. Said competent E. coli cells grow in LB medium of a volume of 5 ml at 37° C. The E. coli cells are shaken at a constant speed of approximately 200 rpm for 16 hours.

Subsequently, 50 ml of LB medium is mixed with 1 ml of the E. coli cells medium and then incubated in a temperature adjustable shaker at a stable condition for approximately 3 to 4 hours or until the bacteria medium has A600 (an absorption reading at a wavelength of 600 nm) of approximately 0.5. Then the mixture is centrifuged at 4° C. at a speed of approximately 12,000 rpm for 5 minutes. The pelleted bacterial cells are collected.

Subsequently, the pellet of the bacterial cells is mixed with approximately 25 ml of CaCl₂ solution of a concentration of approximately 100 mM. The resultant suspension is chilled in iced water for 30 minutes and then shaken at 4° C. with a speed of 12,000 rpm for 5 minutes. The pellet of bacterial cells in CaCl₂ solution (100 mM) is chilled in ice water for 30 minutes and then centrifuged at a speed of approximately 5,000 rpm for 5 minutes at 4° C. Then CaCl₂ solution of a concentration of 100 mM is added to the volume of 2.5 ml and mixed with 30% glycerol. The resultant mixture is chilled in ice water for 60 minutes and collected in tubes of a volume of 1.5 ml.

The mixed type plasmid is mixed with the competent cell and chilled in the ice water for 45 minutes. Then the mixed plasmid is kept in the water storage at 42° C. for 3.30 minutes then chilled in the ice water for 3 minutes. 800 microliter of LB medium is then added before the mixture is then incubated at 37° C. for 1 hour. Then the mixture is centrifuged at 12,000 rpm for 5 minutes.

The supernatant medium is then discarded. The mixture includes approximately 900 microliters of supernatant medium and approximately 100 microliters of the re-suspended pelleted bacterial cells. A glass pipe is used to spread the re-suspended pelleted bacterial cells on the surface of LB medium that has ampicillin of 100 micrograms per milliliter spread on the surface of the LB medium. The bacterial cells are then incubated at 37° C. for 18 hours to 24 hours.

### Clone Selection Using the PCR Reaction

Pick up individual bacterial colonies (which can be seen as white spots on the LB medium) into individual PCR tubes of a size of 0.2 ml. The PCR tubes each contain 5 microliter of distilled water. With each PCR tube, the bacterial colony is mixed with the distilled water and a polymerase chain reaction is performed in association with each PCR tube. The polymerase chain reaction is conducted using techniques, methods, and conditions known in the art. For example, the components of the PCR reaction include a primer with a concentration of 10 pM per ml and a volume of 1.0 microliter 10×PCR solution 1.5 micro liters dNTPs of a concentration of 1 mM 5.0 microliter, Taq polymerase (5 units per microliter) at 0.25 microliter, and water to achieve a total volume of 15 microliters.

The polymerase chain reaction is performed using standard or known program(s) known in the art. For example the polymerase chain reaction includes 1 cycle at 94° C. for 10 minutes, 30 cycles of 94° C. for 1 minute, 60° C. for 1 minute, and 72° C. for 1 minute, followed by 1 cycle of 72° C. for 10 minutes. The result is rechecked by electrophoresis in 0.8% agarose gel, followed by a dying process with ethidium bromide, and detection of UV absorption under UV spectrum.

Extraction of the Recombinant Plasmid from the Clone of E. coli Bacteria by Using the QIAprep Spin Miniprep Kit (QIAGEN)

The specific colony of E. coli bacteria that is determined to include the recombinant plasmid is picked up. More specifically, the colony of E. coli bacteria that includes (or is transformed with) the desired polynucleotide (e.g., the polynucleotide including the gibberellin 20 oxidase gene and the promoter according to various embodiments of the present disclosure) is selected.

The selected colony of E. coli bacteria that has the recombinant plasmid is grown in 50 ml of liquid LB medium that includes 100 mg of ampicillin per ml of LB medium at a temperature of 37° C. in a conical flask. The mixture in the conical flask is shaken at a speed of 200 rpm for a predetermined period of time (e.g., overnight).

The mixture is afterward divided into tubes. Each tube is centrifuged to pellet the bacteria at a speed of 12,000 rpm for 5 minutes. The pellet of bacteria that is produced is collected for use to extract the recombinant plasmid. The extraction of the recombinant plasmid from the pellet can be done using the QIAprep Spin Miniprep kit (QIAGEN) by following the instructions provided by the manufacturer of said testing kit (i.e., QIAprep Spin Miniprep kit (QIAGEN)).

### Analysis of the Polynucleotide Sequence or Base Sequence) of the Recombinant Plasmid

The polynucleotide sequence of the extracted recombinant plasmid can be analysed or determined using known polynucleotide sequencing techniques or method known in the art. For example, the polynucleotide sequence of the recombinant plasmid can be compared to nucleotide sequences (or base sequences) of existing polynucleotides (or DNA/genes) already sequenced, reported, and stored in a known database.

Introduction or Ligation of Promoter into the Plasmid

Cut 35sCaMV promoter from pCAMBIA plasmid. The reaction components includes: 60 microliters of plasmids, 10× buffer at 8 microliters, 1 microliter of BSA, 3 microliters of water, and 8 microliters of enzyme (or restriction enzyme). During the reaction, the reaction mixture or components is incubated at a temperature of 37° C. for 5 hours. Then the reaction mixture, or reaction products, is purified by using QIAquick PCR Purification Kit (QIAGEN) by following the instructions provided by the manufacturer of said QIAquick PCR Purification Kit.

The plasmid is cut by using a suitable enzyme (or restriction enzyme), more specifically a complementary enzyme (or restriction enzyme), for cutting 35sCaMV promoter from the plasmid in the presence of the above-listed reaction components. For example, 30 microliters of plasmid at 10× of 5 microliters of buffer solution, 0.5 microliters of BSA, 10 microliters of water, and 5 microliters of restriction enzyme, are used for cutting the plasmid genome. The reaction occurs over a 5 hour time period at a temperature of approximately 37° C.

The plasmid as cut by the restriction enzyme is put through an electrophoresis process in 1.0% agarose gel. Then the cut plasmid is dyed (e.g., using ethidium bromide) for checking the results. Ethidium bromide binds to DNA molecules, thereby enabling detection of DNA molecules through UV exposure. The plasmid is then cut and extracted from the gel by QIAquick Gel Extraction Kit (QIAGEN) as per the instructions provided by the manufacturer.

Plasmids determined to carry the promoter including a nucleotide sequence of at least 70% sequence similarity to SEQ ID No.2 can be cut using specific restriction enzymes. The reaction components include 60 microliters of plasmid, 10×8 microliters of buffer, 1 microliter of BSA, 2 microliters of water, and 8 microliters of the enzyme. The reaction occurs at a temperature of approximately 37° C. for 5 hours and then the products of the reaction is purified by QIAquick PCR Purification Kit (QIAGEN) using the instructions provided by the manufacturer of said QIAquick PCR purification kit.

The plasmid is using another suitable restriction enzyme using the following reaction components: 30 microliters of plasmid, 10× of microliters of buffer, 0.5 microliters of BSA, 10 microliters of water and 5 microliters of enzyme. The reaction occurs at a temperature of approximately 37° C. for 5 hours.

The plasmid cut by the enzyme is put through an electrophoresis process using 1.0° A agarose gel and then the results are checked using a suitable indicator (e.g., ethidium bromide). The promoter is then cut or extracted from the gel. The extraction of the promoter from the gel can be done by using QIAquick Gel Extraction Kit (QIAGEN) as per the instructions provided by the manufacturer of said QIAquick Gel Extraction Kit (QIAGEN).

The ligation of the promoter to the plasmid pCAMBIA that has the 35sCaMV cut occurs using the following reaction components: approximately 5 microliters of the promoter, approximately 5 microliters of the plasmid pCAMBIA, 10×1 microliter of buffer, 2 microliters of enzyme T4 ligase, and 1 microliter of water. The reaction occurs overnight (or over a predetermined length of time) at a temperature of 4° C. The reaction mixture is then transferred into the E. coli bacteria by using the selective medium that is LB agar including Kanamycin antibiotic at approximately 50 mg per liter. This is incubated for 1 day at a temperature of 37° C. The process enables selection of E. coli colony that includes the plasmid pCAMBIA (or vector) that carries or includes the promoter.

The colony of bacteria that includes the recombinant plasmid (i.e., the plasmid that carries the desired polynucleotide sequences, e.g., the promoter) is collected to grow in 50 ml of LB liquid medium that includes Kanamycin at a concentration of 50 mg per liter. The growth of the bacteria colony occurs over a predetermined period of time (e.g., overnight) at a temperature of 37° C., and with shaking at a speed of approximately 200 rpm. Afterward, the reaction mixture that includes the bacterial cells is spun or centrifuged at the speed of 12,000 rpm for 5 minutes. The pellet of bacteria that is formed is collected for extracting the recombinant plasmid. The extraction of recombinant plasmids can be done using the QIAprep Spin Miniprep kit (QIAGEN) by following the instructions provided by the manufacturer of said testing kit (i.e., QIAprep Spin Miniprep kit (QIAGEN)).

Connection or Ligation of the Gibberellin 20 Oxidase Gene with the Recombinant Plasmid

To clone specific DNA fragments (or polynucleotides) into a plasmid vector, or in other vectors known in the art, the DNA fragments (or polynucleotides) must be produced and then inserted into the vector DNA. Restriction enzymes and DNA ligases are often utilized to produce such recombinant DNA molecules. The cloning of DNA fragments or polynucleotides into plasmid vectors, including connection of the gibberellin 20 oxidase gene with the recombinant plasmid, can occur via methods or techniques that are known in the art, e.g., methods or techniques taught in Lodish et al., Molecular Cell Biology, W. H. Freeman and Company, 2000.

The recombinant plasmid and the gibberellin 20 oxidase gene (or polypeptide encoding for gibberellin 20 oxidase) are both cut by the specific enzyme (e.g., same restriction enzyme) in order to produce complementary polynucleotide ends that can be connected to each other.

The reaction involving the specific enzyme has the components as follows: plasmid, buffer, BSA, and restriction enzyme. The reaction occurs over 2 hours at a temperature of approximately 37° C. The subsequent stopping of the restriction enzyme action can be achieved by increasing the temperature to 65° C. for 10 minutes.

Transformation of Plasmid into Agro-Bacteria

Transformation of plasmid into bacterial cells is a well-known molecular biology procedure or technique, more specifically genetic engineering procedure. For example, agro-bacteria is grown by using solid LB medium and incubated at a temperature of approximately 28° C. for 2 days. A single colony of agro-bacteria is selected to grow in a flask that contains liquid LB medium at a volume of approximately 10-15 ml. The flask is shaken at the speed of 200 rpm at a temperature of approximately 28° C. for 2 days.

Subsequently, argo-bacteria at a volume of 250 ml is added into the flask that contains the liquid LB medium at a volume of 50 ml and the flask is then shaken at the speed of 200 rpm for 14 to 16 hours at a temperature of 28° C. Then the flask is chilled in ice water for 10 minutes. The content of the flask is transferred into tubes and centrifuged at a speed of 6,000 rpm at a temperature of approximately 4° C. for 10 minutes. The pellet of the agro-bacteria that is formed at the base of the tubes is collected by using 10% glycerol solution that is chilled. Initially, each tube has a volume of 25 ml. The tubes are then centrifuged again at a speed of 6,000 rpm at a temperature of approximately 4° C. for 10 minutes. Only the pellets at the base of the bottle is collected by reducing the glycerol to 12.5 ml per tube and then the tube is again centrifuged at the speed of 6,000 rpm, The tube is centrifuged for a third time by reducing the glycerol solution to 0.5 ml. per tube. Then the contents of the tubes are mixed and the centrifugation steps are repeated.

The final pellet obtained via the centrifugation is finally diluted by 10% glycerol that is chilled to a volume of 0.5 ml. Then the 0.5 ml volume of agro-bacterial cells is divided into tubes of a size of 40 microliters. The tubes are stored at a temperature of −80° C. for subsequent use to provide competent agro-bacterial cells for the gene transformed.

Then the competent cells of the agro-bacteria are mixed with the plasmid solution with the volume of 1 microliter and then shaken gently. Afterward, the mix of agro-bacteria and plasmid is chilled in ice water for 2 to 3 minutes. The mix is transferred into cuvettes that are already chilled. After the mix settles at the base of the cuvettes, the cuvettes are dried and kept it in a pulser instrument. An electric technique (e.g., application of electric shock) is used to introduce plasmids into ago-bacterial cells.

The pulser instrument is set at a voltage of 2.5 kV with the capacity of 25 microfarad and a resistance of 600 ohm and the duration is about 4.7 milliseconds. An electric shock is applied to the cuvettes for introducing plasmids into the agro-bacterial cells contained in the cuvettes.

The content of the cuvettes are then transferred into 1.5 ml. tubes. The transformed agro-bacterial cells are then grown at 28° C. The agro-bacterial cells are shaken with the speed of 250 rpm for 1 hour. Then the tubes are centrifuged at 8,000 rpm for 30 seconds. The supernatant bacteria medium present in the tubes after centrifugation is discarded, while keeping the pelleted agro-bacterial cells. The pelleted bacterial cells are re-suspended. A glass pipe is used to spread the diluted or resuspended agro-bacterial pellet onto the surface of LB medium that includes the Kanamycin antibiotic at 50 mg per ml. The LB medium with agro-bacterial cells spread thereon is incubated at 28° C. for 2 days to allow growth of colonies of agro-bacterial cells.

After two days, individual colonies of agro-bacterial cells (which are present as white spots in the surface of LB medium) are transferred into individual PCR tubes of a size of 0.2 ml. Each tube includes approximately 5 microliters of distilled water. PCR. Is used to check the results. The PCR reaction occurs via known techniques, methods, and conditions. For example PCR reaction components include primer at a concentration of 10 pM per ml, 1.0 microliter of 10×PCR solution, 1.5 microliters of dNTPs at 1 mM, 5.0 microliters Taq polymerase (5 units per micro liter) 0.25 microliter, and water to achieve a total volume of 15 microliter. The PCR occurs via a standard PCR program, for example including 1 cycle of 94° C., 30 cycles of 94° C. for 1 minute, 60° C. for 1 minute, and 72° C. for 1 minute, and another. 1 cycle at 72° C. for 10 minutes. The results of the PCR are checked by electrophoresis using 0.8% agarose gel and ethidium bromide dye. DNA molecules can be detected via UV application.

### Plant (e.g., Tobacco Plant) Transformation

Polynucleotides provided by various embodiments of the present disclosure are introduced or inserted into plants, plant cells, or plant tissues in a process known as plant transformation. The introduction of foreign polynucleotides (i.e., polynucleotides that do not occur naturally in said plant) into the genome of a plant results in production of a transgenic plant. Polynucleotides can be introduced into plants, plant cells, or plant tissues (i.e., plant transformation) can occurs via known techniques or methods in the art.

Techniques for transformation processes include agrobacteriatransfection, electroporation, microinjection, transduction, cell fusion, dextran, calcium phosphate precipitation, lipofection, gene transferring gun, transporter for transferring DNA vectors, DNA injection, use of viruses, or via micro projection.

In an embodiment of the present disclosure, agro-bacteria can be used for transforming plant cells or tissues (i.e., to introduce foreign polynucleotides or genes into plant genome). The transformation technique is applicable with zygotic or somatic or other plant cells or tissues, for instance cotyledon, seed, leaf, root, or stem cells/tissues.

A tobacco plant is grown in sterile conditions that include a solid synthetic medium that does not include any chemicals that might influence the growth rate of the tobacco plant. The tobacco plant is exposed to light for 16 hours per day at a temperature of 25° C.±2° C. over a period of 2 weeks until the tobacco plant is mature.

Agro-bacteria that includes the recombinant plasmid including the Gibberellin 20 oxidase gene and the promoter provided by various embodiments of the present disclosure is grown in a solid LB medium. The medium includes Kanamycin antibiotic at a concentration of 50 milligrams per liter. The LB medium with agro-bacteria cultivated thereon is incubated at a temperature of approximately 28° C. for 2 days.

A single colony can be collected to grow in liquid LB medium with Kanamycin at a concentration of 50 mg per liter. The LB medium including the colony of agro-bacteria is shaken at a speed of 200 rpm at a temperature of approximately 28° C. for 14 to 16 hours. A spectrum absorption instrument is then used to measure the spectrum absorption index at a wavelength of 600 nanometers (OD600). The agrobacteria is grown in the LB medium until the spectrum absorption index of said LB medium is between approximately 1.0 and 1.2. Then, the LB medium is centrifuged to collect the pelleted agro-bacterial cells. Centrifugation occurs at a speed of 5,000 rpm at a temperature of 4° C. for 10 minutes.

The pellet of agro-bacteria is then collected by using the B5 liquid medium that is added NAA 0.02 mg. per liter about 50 milliliters and added acetosyringone at a concentration of 100 micromole per liter for use in transformation of the tobacco plant.

Transformation of the tobacco plant is done by using the agro-bacteria. The center of the leaves of the tobacco plant is cut with a square shape at the size of 1 sq·cm. Then, the leaves are kept in a bottle to grow the ago-bacteria therein. The agro-bacteria includes the recombinant plasmid which includes the Gibberellin 20 oxidase gene and the promoter. Approximately 25 leaves of the tobacco plant are used per approximately 25 milliliters of the solution. Then the solution is shaken at a speed of 100 rpm for 30 minutes. Afterward, the tobacco leaves are dried using tissue paper and then the leaves are grown in a MS medium that has a sugar level of 20 grams per liter IAA 0.05 milligrams per liter BAP 2.0 milligrams per liter and agar powder 0.85% pH 5.7 for 2 days with cefotaxime at a concentration of 500 mg per liter and a selective hygromycin B at a concentration of 50 mg per liter. The solution is exposed to light for 16 hours per day at a temperature of approximately 25° C.±2° C. The medium is changed every 2 weeks until the formation of transgenic tobacco plant (i.e., until transformation of the tobacco plant is completed).

Transferring of vectors (or plasmids) into plant cells or plant tissues by a transformation technique is applicable with plant cell and plant tissues that clonally grow in either organogenesis or embryogenesis. The transformation technique or process can be applied to many types of plant cells or tissue, depending upon the system of clonal tissue separation in plants. The examples of the suitable tissues are pollen, leaves, embryo, cotyledon, hypocotyl, megametophyte, callus tissue, or meristem tissue.

The process of gene transfer into cells can be either permanent or temporary. In addition, the process of gene transfer can use either vectors with independent separation or vectors that combine external gene into the plant genome. After gene transfer, the plant cells or tissues can grown by using various techniques or method known in the art, for example growing the plant cells or tissue in liquid culture medium.

### Determination or Examination of the Existence of the Gibberellin 20 Oxidase Gene and the Promoter in a Tobacco Plant

The DNA of the tobacco leaves that are expected to be transformed (or transgenic) are examined for the presence of the gibberellin 20 oxidase gene and the promoter by using PCR techniques known in the art. For instance, primer at a concentration of 10 pM per liter with a volume of 1.0 microliter, 10×PCR solution at 1.5 microliters, and 5.0 microliters of dNTPs of a concentration of 1 mM, 0.2 microliters Taq polymerase (5 units per microliters), and 4.8 microliters of water. 1 microliter of the prototyped DNA to achieve a total volume of 15 microliters is also included for the PCR. PCR is performed using standard conditions, for example 1 cycle of 94° C. for 10 minutes, 30 cycles of 94° C. for 1 minute, 72° C. for 1 minute, and 60° C. for 1 minute 30 seconds for 30 cycles, and 1 cycle of 72° C. for 10 minutes. The results are then examined by electrophoresis using 0.8% agarose gel before application of a dye of ethidium bromide. Ethidium bromide binds to DNA molecules thereby enabling detection of said DNA molecules via UV absorption techniques.

### Examination and Measurement of Plant Growth

Plant growth (e.g., length or diameter of plants) was measured at predetermined time intervals to thereby determine characteristics, for example rate, of plant growth. The growth of plants that have been genetically modified (i.e., transgenic plants of various embodiments of the present disclosure) as well as non-transgenic or wild-type plants were measured in order to compare the differences in plant growth between the transgenic plants and the wild-type plants.

To measure the growth of the plant, the height of the plant at predetermined time periods is measured. For instance, 3 generations of transgenic plants (with 3 plants per generation) and 3 generations of wild-type plants (with 3 plants per generation) were used, and the heights of each of said plants were measured at fixed time intervals, for example each day. The collected plant height data is then used to analyze statistical changes or patterns that occur to thereby evaluate plant growth characteristics, for example rate of plant growth.

### Examination and Measurement of Fiber Production (or Cellulose Yield)

The fiber content of a plant is determined for measuring or determining cellulose yield of the plant. To determine the fiber content of a plant, the plant is first cut into small pieces. More specifically, a selected sample of the plant is cut into small pieces. The plant pieces were macerated by boiling. A chemical, for example Na₂O at a concentration of 100 g.AA Na₂O per liter is used for boiling the plant pieces. Alternatively, hydrogen peroxide and glacial acetic acid can be used to boil the plant pieces. The boiling process occurred with mixing for 1 minute at a pressure of approximately 12 to 14 AMP.

Subsequently, plant tissue was separated from the chemical (which appeared as a black liquor) and collected by rinsing the plant tissue with water using a sieve with a pore size of 200. The plant tissue is rinsed using the sieve until no chemical is left. The pore size of the sieve is too small to allow the tissue to be displaced through the sieve, thereby trapping and separating the plant tissue.

Then the tissue is shaken for 10 minutes before being mixed with water to get a dispersion of about 0.2%. The productivity of the tissue is calculated using methods or techniques that are known in the art. For example, a fiber analyzer (KAJAANI FiberLab, Valmet Automation Kajaani Ltd. Kajaani, Finland) is used for measuring fiber production (e.g., fiber length) and according cellulose yield of the plant. Other techniques for evaluating, measuring, or determining various cellulose characteristics of plants are described in James F. Beecher, Christopher G. Hunt, and J. Y. Zhu, “Tools for the Characterization of Biomass at the Nanometer Scale”, and Park et al., Cellulose crystallinity index: measurement techniques and their impact on interpreting cellulase performance, Biotechnology for Biofuels, 3: 10, 2010.

### Results

### Example One

The heights of a set of transgenic plants, which are plants that include the gibberellin 20 oxidase gene and the promoter, and a set of wild-type plants (control plants), which are plants that do not include the gibberellin 20 oxidase gene or the promoter are measured.

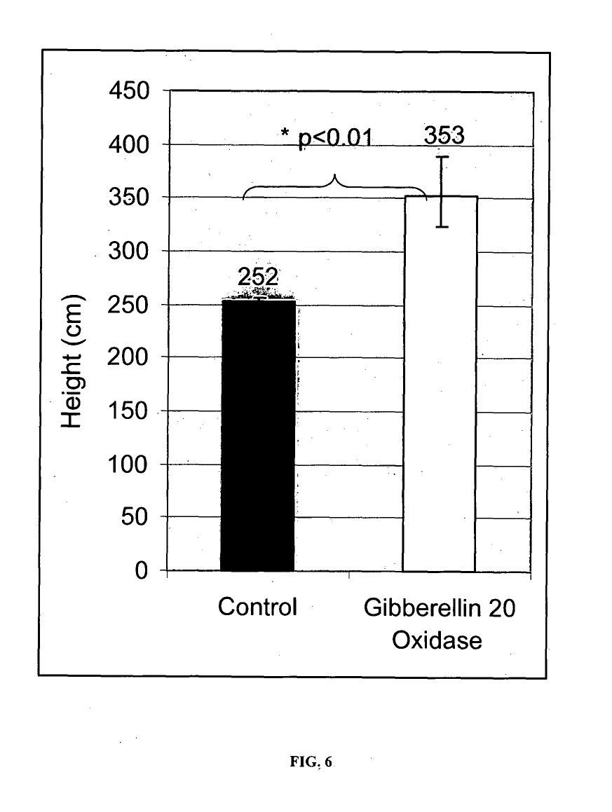

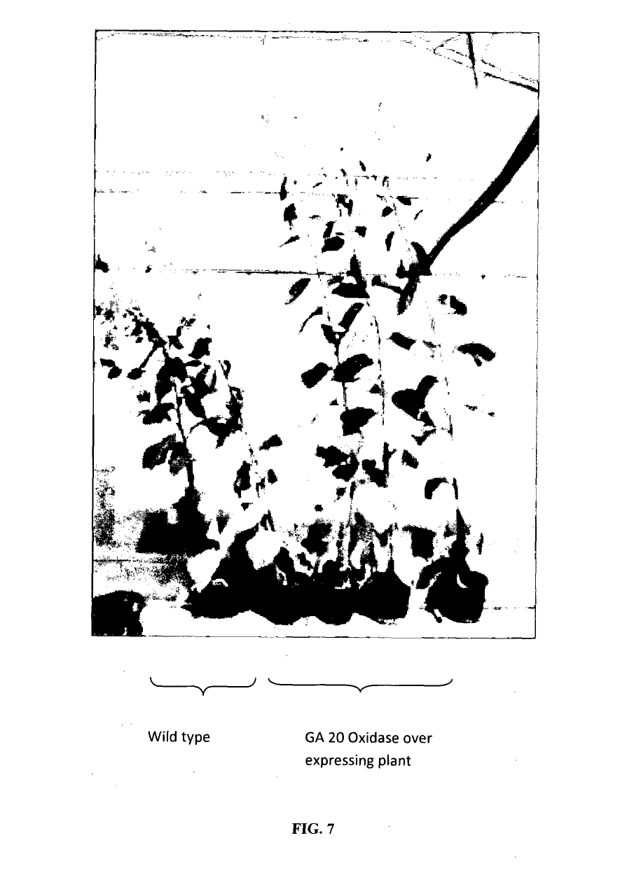

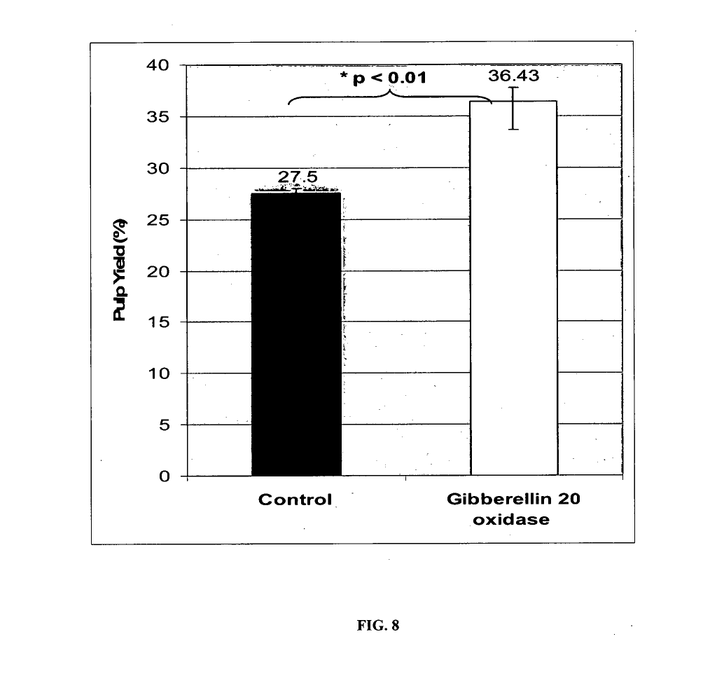

FIG. 6 shows the average heights of the transgenic plants as compared to the average heights of the wild-type plants (or control plants). FIG. 7 is a picture of a number (or set) of transgenic plants and a number (or set) of wild-type plants.

It was found that, measured when both transgenic plants and the wild type plants are of the same age, the average height of the transgenic plants is 252 cm and the average height of the wild type plants is 353 cm. This shows that the transgenic plants have a height increase of approximately 40.1% over the wild-type plants.

The increase in height can attributed to an increased expression of the gibberellin 20 oxidase gene, and consequent production of gibberellin 20 oxidase, due to the presence and expression of the promoter. The promoter causes, directs, facilitates, or effectuates an increased expression of the gibberellin 20 oxidase gene, and consequent production of gibberellin 20 oxidase.

In addition, the height increase observed with transgenic plants as compared to wild-type plants represents an acceleration of plant growth in transgenic plants as compared to wild-type plants. The promoter can facilitate or effectuate an increased expression of the gibberellin 20 oxidase gene, and consequent production of gibberellin 20 oxidase, thereby resulting in an acceleration in plant growth in transgenic plants.

The approximately 40.1% increase in the average height of transgenic plants as compared to wild-type plants is a significant, unexpected, and/or surprising result associated with the expression of the gibberellin 20 oxidase gene together with the promoter as according to several embodiments of the present disclosure.

### Example Two

The cellulose production, and accordingly pulp yield, of a set of transgenic plants, which are plants that include the gibberellin 20 oxidase gene and the promoter, and a set of wild-type plants (control plants), which are plants that do not include the gibberellin 20 oxidase gene or the promoter are measured. The cellulose production, and accordingly pulp yield, is measured based on the ratio of cellulose amount per total wood. Therefore, an increase in cellulose production, and hence pulp yield, does not arise from an increase of plant growth.

FIG. 8 shows the relative average pulp yield between the set of transgenic plants and the set of wild-type (or control) plants. The average pulp yield of the wild-type plants is approximately 27.5% and the average pulp yield of the transgenic plants is approximately 36.43%. The results shows that the transgenic plants have an average increased pulp yield of approximately 32.5% as compared to the wild-type plants. The increase in average pulp yield associated with transgenic plants as compared to wild-type plants is a significantly, surprising, and unexpected result.

The results presented in FIG. 8 also suggest that the promoter is capable of directing, facilitating, or effectuating specific expression of the gibberellin 20 oxidase gene in plant vascular tissue. The promoter can facilitate or effectuate specific increased in cellulose production. The results suggest that the promoter of certain embodiments enables increased expression of gibberellin 20 oxidase gene, and consequent production of gibberellin 20 oxidase specifically in vascular tissue to thereby increase the cellulose production in said transgenic plant. The ability of the promoter to specifically direct expression of gibberellin oxidase gene, and consequent production of gibberellin 20 oxidase, in plant vascular tissue (e.g., xylem tissue) is significant, surprising, and/or unexpected. In addition, the ability of the promoter to specifically facilitate or effectuate an increased expression of gibberellin oxidase gene, and consequent production of gibberellin 20 oxidase, in plant vascular tissue (e.g., xylem tissue) is significant, surprising, and/or unexpected.

### Example Three

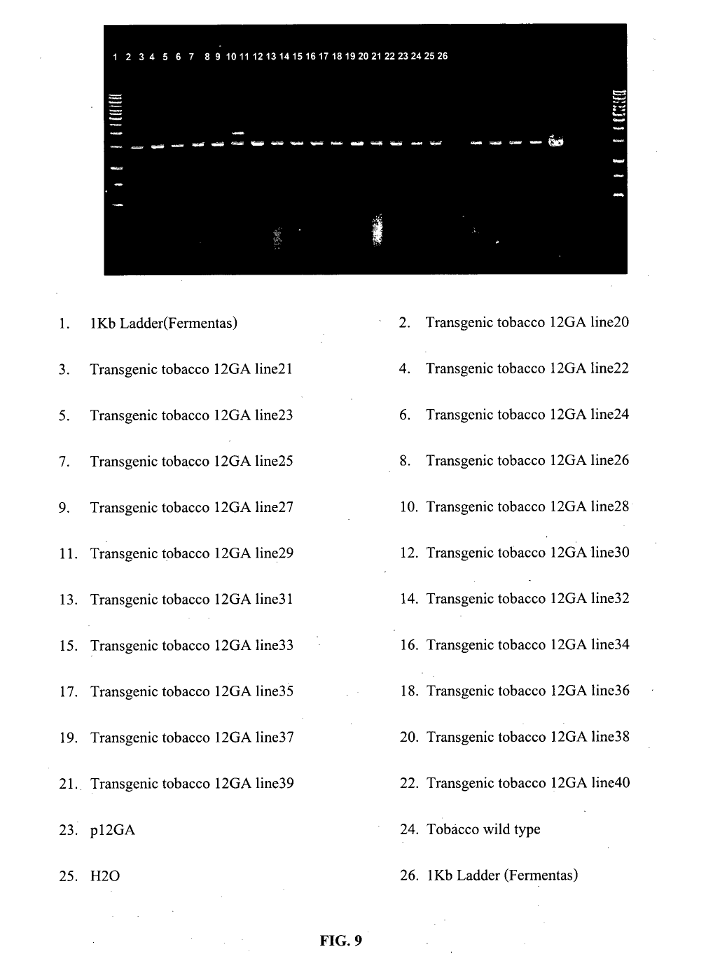

FIG. 9 shows a picture from a gel electrophoresis test. 21 DNA samples obtained from transgenic tobacco plants were used (or run) in lanes 2 to 22, a p 12GA sample (i.e., plasmid vector including the gibberellin 20 oxidase gene and the promoter) was run in lane 23, and two controls, namely a sample from a wild-type tobacco plant and a water sample, were used (or run) in lanes 24 and 25. Lanes 1 and 26 are loaded with 1 kb Ladder (Fermentas).

The results shown in FIG. 9 indicate that the transgenic tobacco plants include the gibberellin 20 oxidase gene as well as the promoter. In addition, the results shown in FIG. 9 indicate that the wild type tobacco plant and water do not include the gibberellin 20 oxidase gene or promoter.

The present disclosure provides polynucleotides for increasing the production of gibberellin 20 oxidase. In several embodiments, the polynucleotides increase the production of gibberellin 20 oxidase specifically in plant vascular tissue (e.g., xylem tissue). The polynucleotides of many embodiments includes a gibberellin 20 oxidase gene that includes at least one of (a) a nucleotide sequence of SEQ ID No. 2 as shown in FIG. 3; (b) a nucleotide sequence of substantial sequence similarity to SEQ ID No. 2; (c) a nucleotide sequence that complements or is able to hybridize to (a) or (b); or (d) a nucleotide sequence which is the reverse complement of (a), (b), or (c) as well as a promoter (or promoter sequence) that includes at least one of (1) a nucleotide sequence listed in SEQ ID No. 1; (2) a nucleotide sequence of substantial sequence similarity to SEQ ID No. 1; (3) a nucleotide sequence that complements or is able to hybridize to (1) or (2); and (4) a nucleotide sequence which is the reverse complement of (1), (2), or (3)

The promoter modulates the expression of the gibberellin 20 oxidase gene. In several embodiments, the promoter facilitates or effectuates specific expression of the gibberellin 20 oxidase gene, and consequent production of gibberellin 20 oxidase, in plant vascular tissue (e.g., xylem tissue). This specific expression of the gibberellin 20 oxidase gene, and consequent production of gibberellin 20 oxidase, in plant vascular tissue as disclosed by several embodiments of the present disclosure, is significant, unexpected, and/or surprising.

In some embodiments, the promoter increases the expression of gibberellin 20 oxidase gene to thereby increase the production of gibberellin 20 oxidase. The increase in expression of the gibberellin 20 oxidase gene, and consequent increase in production of gibberellin 20 oxidase is significant, surprising, and/or unexpected. In various embodiments, the promoter facilitates or effectuates an increase in expression of the gibberellin 20 oxidase gene, and consequent production of gibberellin 20 oxidase by at least 10%. In specific embodiments, the promoter facilitates or effectuates an increase in expression of the gibberellin 20 oxidase gene, and consequent production of gibberellin 20 oxidase by at least more specifically at least approximately 25%, and even by at least approximately 40%.

Several embodiments of the present disclosure also provide transgenic plants that include the polynucleotide of various embodiments of the present disclosure. More specifically, the transgenic plants include the gibberellin 20 oxidase gene for expressing gibberellin 20 oxidase, as well as the promoter for modulating expression of the gibberellin 20 oxidase gene. The transgenic plant can demonstrate increased plant growth, or accelerated plant growth, as compared to wild type plants (i.e., plants without the gibberellin 20 oxidase gene or the promoter). The increase, or acceleration, in plant growth is due to the combined, or mutually dependent, expression of the promoter and the gibberellin 20 oxidase gene. The increase, or acceleration, in plant growth in some embodiments is at least approximately 10%, and in various embodiments at least approximately 25%. The increase significant, or acceleration, in plant growth is significant, unexpected, and/or surprising. The transgenic plant can demonstrate increased cellulose production and yield as compared to wild type plants. The increase, or acceleration, in plant growth is due to the combined, or mutually dependent, expression of the promoter and the gibberellin 20 oxidase gene. The increase, or acceleration, in cellulose production and yield in some embodiments is at least approximately 10%, and in various embodiments at least approximately 25%. The increase significant, or acceleration, in cellulose production and yield associated with transgenic plants is significant, unexpected, and/or surprising.

Particular embodiments of the disclosure are described above for addressing at least one of the previously indicated problems. While features, functions, processes, process portions, advantages, and alternatives associated with certain embodiments have been described within the context of those embodiments, other embodiments may also exhibit such advantages, and not all embodiments need necessarily exhibit such advantages to fall within the scope of the disclosure. It will be appreciated that several of the above-disclosed features, functions, processes, process portions, advantages, and alternatives thereof, may be desirably combined into other different methods, processes, systems, or applications. The above-disclosed features, functions, processes, process portions, or alternatives thereof, as well as various presently unforeseen or unanticipated alternatives, modifications, variations or improvements thereto that may be subsequently made by one of ordinary skill in the art, are encompassed by the following claims.

1. A polynucleotide comprising a gibberellin 20 oxidase gene and a promoter, the promoter comprising one of:
(a) a nucleotide sequence of SEQ ID No. 2;
(b) a nucleotide sequence of at least 70% sequence similarity to SEQ ID No. 2;
(c) a nucleotide sequence that is able to hybridize to at least one of (a) and (b); and
(d) a nucleotide sequence which is the reverse complement of at least one of (a), (b), and (c).

2. The polynucleotide as in claim 1, wherein the promoter comprises a nucleotide sequence of at least 80% sequence similarity to SEQ ID No. 2.

3. The polynucleotide as in claim 2, wherein the promoter comprises a nucleotide sequence of at least 90% sequence similarity to SEQ ID No. 2.

4. The polynucleotide as in claim 1, wherein the promoter is obtained from a Eucalypt plant.

5. The polynucleotide as in claim 4, wherein the promoter is obtained from a Eucalyptus plant.

6. The polynucleotide as in claim 1, wherein the gibberellin 20 oxidase gene comprises one of:
(1) a nucleotide sequence as listed in SEQ ID No. 1;
(2) a nucleotide sequence of at least 70% sequence similarity to SEQ ID No. 1;
(3) a nucleotide sequence that is able to hybridize to at least one of (1) and (2); and
(4) a nucleotide sequence which is the reverse complement of at least one of (1), (2), and (3).

7. The polynucleotide as in claim 6, wherein the gibberellin 20 oxidase gene comprises a nucleotide sequence of at least 80% sequence similarity to SEQ ID No. 1.

8. The polynucleotide as in claim 7, wherein the gibberellin 20 oxidase gene comprises a nucleotide sequence of at least 90% sequence similarity to SEQ ID No. 1.

9. The polynucleotide as in claim 1, wherein the promoter is a vascular specific promoter that one of facilitates and effectuates specific expression of the gibberellin 20 oxidase gene in plant vascular tissue.

10. The polynucleotide as in claim 1, wherein expression of the gibberellin oxidase gene produces gibberellin 20 oxidase polypeptide comprising one of:
(i) an amino acid sequence of SEQ ID No. 3;
(ii) an amino acid sequence of at least 70% sequence similarity to SEQ ID No. 3; and
(iii) an amino acid sequence that is able to hybridize to at least one of (i) or (ii).

11. The polynucleotide as in claim 10, wherein the gibberellin 20 oxidase polypeptide comprises an amino acid sequence of at least 80% sequence similarity of SEQ ID No. 3.

12. The polynucleotide as in claim 11, wherein the gibberellin 20 oxidase polypeptide comprises an amino acid sequence of at least 90% sequence similarity of SEQ ID No. 3.

13. The polynucleotide as in claim 1, wherein the promoter one of facilitates and effectuates an increased expression of gibberellin oxidase gene and consequent production of gibberellin 20 oxidase polypeptide by at least approximately 10%.

14. The polynucleotide as in claim 13, wherein the promoter one of facilitates and effectuates an increased expression of gibberellin oxidase gene and consequent production of gibberellin 20 oxidase polypeptide by at least approximately 25%.

15. The polynucleotide as in claim 14, wherein the vascular specific promoter one of facilitates and effectuates an increased expression of gibberellin oxidase gene and consequent production of gibberellin 20 oxidase by at least approximately 40%.

16. The polynucleotide as in claim 13, wherein increased expression of gibberellin 20 oxidase gene and production of gibberellin 20 oxidase polypeptide effectuates at least one of accelerated plant growth and enhanced cellulose production.

17. The polynucleotide as in claim 16, wherein the increased expression of gibberellin 20 oxidase gene and production of gibberellin 20 oxidase polypeptide effectuates an at least 20% acceleration in plant growth.

18. The polynucleotide as in claim 16, wherein the increased expression of gibberellin 20 oxidase gene and production of gibberellin 20 oxidase polypeptide effectuates an at least 20% increase in cellulose production.

19. A vector carrying a polynucleotide that comprises a gibberellin 20 oxidase gene for expressing gibberellin 20 oxidase polypeptide and a promoter for controlling the expression of the gibberellin 20 oxidase gene, wherein the promoter comprises one of:
(a) a nucleotide sequence of SEQ ID No. 2;
(b) a nucleotide sequence of at least 70% sequence similarity to SEQ ID No. 2;
(c) a nucleotide sequence that is able to hybridize to at least one of (a) and (b); and
(d) a nucleotide sequence which is the reverse complement of at least one of (a), (b), and (c).

20. The vector as in claim 19, wherein the promoter comprises a nucleotide sequence of at least 80% sequence similarity to SEQ ID No. 2.

21. The vector as in claim 20, wherein the promoter comprises a nucleotide sequence of at least 90% sequence similarity to SEQ ID No. 2.

22. The vector as in claim 19, wherein the promoter is obtained from a Eucalypt plant.

23. The vector as in claim 22, wherein the promoter is obtained from a Eucalyptus plant.

24. The vector as in claim 19, wherein the gibberellin 20 oxidase gene comprises one of:
(1) a nucleotide sequence listed in SEQ ID No. 1;
(2) a nucleotide sequence of at least 70% sequence similarity to SEQ ID No. 1;
(3) a nucleotide sequence that is able to hybridize to at least one of (1) and (2); and
(4) a nucleotide sequence which is the reverse complement of at least one of (1), (2), and (3).

25. The vector as in claim 24, wherein the gibberellin 20 oxidase gene comprises a nucleotide sequence of at least 80% sequence similarity to SEQ ID No. 1.

26. The vector as in claim 25, wherein the gibberellin 20 oxidase gene comprises a nucleotide sequence of at least 90% sequence similarity to SEQ ID No. 1.

27. The vector as in claim 19, wherein the promoter is a vascular specific promoter that one of facilitates and effectuates specific expression of the gibberellin 20 oxidase gene in plant vascular tissue.

28. The vector as in claim 27, wherein the vascular specific promoter one of facilitates and effectuates an increased expression of gibberellin 20 oxidase gene and consequent production of gibberellin 20 oxidase by at least approximately 10%.

29. The vector as in claim 28, wherein the vascular specific promoter one of facilitates and effectuates an increased expression of gibberellin 20 oxidase gene and consequent production of gibberellin 20 oxidase by at least approximately 25%.

30. A transgenic plant cell comprising a polynucleotide that comprises a gibberellin oxidase gene for expressing a gibberellin 20 oxidase polypeptide and a promoter for controlling expression of the gibberellin oxidase gene, wherein the promoter comprises one of:
(a) a nucleotide sequence of SEQ ID No. 2;
(b) a nucleotide sequence of at least 70% sequence similarity to SEQ ID No. 2;
(c) a nucleotide sequence that is able to hybridize to at least one of (a) and (b); and
(d) a nucleotide sequence which is the reverse complement of at least one of (a), (b), and (c).

31. The transgenic plant cell as in claim 30, wherein the promoter comprises a nucleotide sequence of at least 80% sequence similarity to SEQ ID No. 2.

32. The transgenic plant cell as in claim 31, wherein the promoter comprises a nucleotide sequence of at least 90% sequence similarity to SEQ ID No. 2.

33. The transgenic plant cell as in claim 30, wherein the promoter is obtained from a Eucalypt plant.

34. The transgenic plant cell as in claim 33, wherein the promoter is obtained from a Eucalyptus plant.

35. The transgenic plant cell as in claim 30, wherein the gibberellin 20 oxidase gene comprises one of:
(1) a nucleotide sequence as listed in SEQ ID No. 1;
(2) a nucleotide sequence of at least 70% sequence similarity to SEQ ID No. 1;
(3) a nucleotide sequence that is able to hybridize to at least one of (1) and (2); and
(4) a nucleotide sequence which is the reverse complement of at least one of (1), (2), and (3).

36. The transgenic plant cell as in claim 35, wherein the gibberellin 20 oxidase gene comprises a nucleotide sequence of at least 80% sequence similarity to SEQ ID No. 1.

37. The transgenic plant cell as in claim 36, wherein the gibberellin 20 oxidase gene comprises a nucleotide sequence of at least 90% sequence similarity to SEQ ID No. 1.

38. The transgenic plant cell as in claim 30, wherein the gibberellin 20 oxidase polypeptide comprises one of:
(i) an amino acid sequence of SEQ ID No. 3;
(ii) an amino acid sequence of at least 70% sequence similarity to SEQ ID No. 3; and
(iii) an amino acid sequence that is able to hybridize to at least one of (i) and (ii).

39. The transgenic plant cell as in claim 38, wherein gibberellin 20 oxidase polypeptide comprises an amino acid sequence of at least 80% sequence similarity to SEQ ID No. 3.

40. The transgenic plant cell as in claim 39, wherein gibberellin 20 oxidase polypeptide comprises an amino acid sequence of at least 90% sequence similarity to SEQ ID No. 3.

41. The transgenic plant cell as in claim 30, wherein the promoter is a vascular specific promoter that one of facilitates and effectuates specific expression of the gibberellin 20 oxidase gene in plant vascular tissue.

42. The transgenic plant cell as in claim 41, wherein the vascular specific promoter one of facilitates and effectuates an increase in expression of gibberellin 20 oxidase gene and consequent production of gibberellin 20 oxidase polypeptide by at least approximately 10%.

43. The transgenic plant cell as in claim 42, wherein the vascular specific promoter one of facilitates and effectuates an increase in expression of gibberellin 20 oxidase gene and consequent production of gibberellin 20 oxidase polypeptide by at least approximately 25%.

44. The transgenic plant cell as in claim 43, wherein the vascular specific promoter one of facilitates and effectuates an increase in expression of gibberellin 20 oxidase gene and consequent production of gibberellin 20 oxidase polypeptide by at least approximately 40%.

45. The transgenic plant cell as in claim 30, wherein said transgenic plant cell is from a perennial plant.

46. The transgenic plant cell as in claim 45, wherein the transgenic plant cell is from one of a Eucalyptus plant and a Tobacco plant.

47. A process for generating a transgenic plant cell via a gene transformation technique using an agro-bacteria to transfer a polynucleotide into a plant cell, wherein the polynucleotide comprises a gibberellin 20 oxidase gene and a promoter, the promoter comprising a nucleotide sequence of at least 70% sequence similarity to SEQ ID No. 2.

48. The process as in claim 47, wherein the promoter comprises a nucleotide sequence of at least 80% sequence similarity to SEQ ID No. 2.

49. The process as in claim 48, wherein the promoter comprises a nucleotide sequence of at least 90% sequence similarity to SEQ ID No. 2.

50. The process as in claim 47, wherein the promoter is obtained from a Eucalypt plant.

51. The process as in claim 50, wherein the promoter is obtained from a Eucalyptus plant.

52. The process as in claim 47, wherein the gibberellin 20 oxidase gene comprises a nucleotide sequence of at least 70% sequence similarity to SEQ ID No. 1.

53. The process as in claim 52, wherein the gibberellin 20 oxidase gene comprises a nucleotide sequence of at least 80% sequence similarity to SEQ ID No. 1.

54. The process as in claim 53, wherein the gibberellin 20 oxidase gene comprises a nucleotide sequence of at least 90% sequence similarity to SEQ ID No. 1.

55. The process as in claim 47, wherein the promoter is a vascular specific promoter that one of facilitates and effectuates expression of the gibberellin 20 oxidase gene in plant vascular tissue.

56. The process as in claim 55, wherein the vascular specific promoter increases the expression of the gibberellin 20 oxidase gene and consequent production of gibberellin 20 oxidase polypeptide in plant vascular tissue by at least 25%.

57. The process as in claim 52, wherein the transgenic plant cell is from a perennial plant.

58. The process as in claim 57, wherein the transgenic plant cell is from one of a Eucalyptus plant and a Tobacco plant.

59. A process for modulating plant growth comprising:
transfecting a plant with a polynucleotide that comprises a gibberellin 20 oxidase gene and a promoter that comprises a nucleotide sequence of at least 70% sequence similarity to SEQ ID No. 2; and
expressing the gibberellin 20 oxidase gene in the plant to produce gibberellin 20 oxidase polypeptide.

60. The process as in claim 59, wherein the promoter comprises a nucleotide sequence of at least 80% sequence similarity to SEQ ID No. 2.

61. The process as in claim 59, wherein the promoter is obtained from a Eucalypt plant.

62. The process as in claim 61, wherein the promoter is obtained from a Eucalyptus plant.

63. The process as in claim 60, expressing the gibberellin 20 oxidase gene in the plant cell comprising:
directing production of gibberellin 20 oxidase polypeptide specifically to plant vascular tissue.

64. The process as in claim 63, expressing the gibberellin 20 oxidase gene in the plant cell further comprising:
using the promoter to increase the production of gibberellin 20 oxidase polypeptide by at least approximately 10%,
wherein the increased production of gibberellin 20 oxidase polypeptide one of facilitates and effectuates at least one of acceleration of plant growth and increase in cellulose yield of the plant.

65. The process as in claim 64, wherein the increase in the production of gibberellin 20 oxidase polypeptide is at least approximately 25%.

66. The process as in claim 65, wherein the increase in the production of gibberellin 20 oxidase polypeptide is at least approximately 40%.

67. The process as in claim 64, wherein the gibberellin 20 oxidase polypeptide comprises an amino acid sequence of at least 70% sequence similarity to SEQ ID No. 3.

68. The process as in claim 64, wherein the gibberellin 20 oxidase gene comprises a nucleotide sequence of at least 70% sequence similarity to SEQ ID No. 2.

69. The process as in claim 68, wherein the gibberellin 20 oxidase gene comprises a nucleotide sequence of at least 80% sequence similarity to SEQ ID No. 2.

70. The process as in claim 69, wherein the plant is a perennial plant.

71. The process according to claim 70, wherein the perennial plant is one of a Eucalyptus plant and a Tobacco plant.

72. A transgenic perennial plant comprising a polynucleotide that comprises a gibberellin oxidase gene for expressing a gibberellin 20 oxidase polypeptide and a promoter for controlling expression of the gibberellin oxidase gene, wherein the promoter comprises one of:
(a) a nucleotide sequence of SEQ ID No. 2;
(b) a nucleotide sequence of at least 70% sequence similarity to SEQ ID No. 2;
(c) a nucleotide sequence that is able to hybridize to at least one of (a) and (b); and
(d) a nucleotide sequence which is the reverse complement of at least one of (a), (b), and (c).

73. The transgenic perennial plant as in claim 72, wherein the promoter comprises a nucleotide sequence of at least 80% sequence similarity to SEQ ID No. 2.

74. The transgenic perennial plant as in claim 73, wherein the promoter comprises a nucleotide sequence of at least 90% sequence similarity to SEQ ID No. 2.

75. The transgenic perennial plant as in claim 72, wherein the promoter is obtained from a Eucalypt plant.

76. The transgenic perennial plant as in claim 75, wherein the promoter is obtained from a Eucalyptus plant.

77. The transgenic perennial plant as in claim 72, wherein the gibberellin 20 oxidase gene comprises one of:
(1) a nucleotide sequence as listed in SEQ ID No. 1;
(2) a nucleotide sequence of at least 70% sequence similarity to SEQ ID No. 1;
(3) a nucleotide sequence that is able to hybridize to at least one of (1) and (2); and
(4) a nucleotide sequence which is the reverse complement of at least one of (1), (2), and (3).

78. The transgenic perennial plant as in claim 77, wherein the gibberellin 20 oxidase gene comprises a nucleotide sequence of at least 80% sequence similarity to SEQ ID No. 1.

79. The transgenic perennial plant as in claim 78, wherein the gibberellin 20 oxidase gene comprises a nucleotide sequence of at least 90% sequence similarity to SEQ ID No. 1.

80. The transgenic perennial plant as in claim 72, wherein the gibberellin 20 oxidase polypeptide comprises one of:
(i) an amino acid sequence of SEQ ID No. 3;
(ii) an amino acid sequence of at least 70% sequence similarity to SEQ ID No. 3; and
(iii) an amino acid sequence that is able to hybridize to at least one of (i) and (ii).

81. The transgenic perennial plant as in claim 80, wherein gibberellin 20 oxidase polypeptide comprises an amino acid sequence of at least 80% sequence similarity to SEQ ID No. 3.

82. The transgenic perennial plant as in claim 81, wherein gibberellin 20 oxidase polypeptide comprises an amino acid sequence of at least 90% sequence similarity to SEQ ID No. 3.

83. The transgenic perennial plant as in claim 72, wherein the promoter is a vascular specific promoter that one of facilitates and effectuates specific expression of the gibberellin 20 oxidase gene in plant vascular tissue.

84. The transgenic perennial plant as in claim 83, wherein the vascular specific promoter one of facilitates and effectuates an increase in expression of gibberellin 20 oxidase gene and consequent production of gibberellin 20 oxidase polypeptide by at least approximately 10%.

85. The transgenic perennial plant as in claim 84, wherein the vascular specific promoter one of facilitates and effectuates an increase in expression of gibberellin 20 oxidase gene and consequent production of gibberellin 20 oxidase polypeptide by at least approximately 25%.

86. The transgenic perennial plant as in claim 85, wherein the vascular specific promoter one of facilitates and effectuates an increase in expression of gibberellin 20 oxidase gene and consequent production of gibberellin 20 oxidase polypeptide by at least approximately 40%.

87. The transgenic perennial plant as in claim 83, wherein the transgenic perennial plant is one of a Eucalyptus plant and a Tobacco plant.
